(19) United States
(12) Patent Application Publication
(10) Pub. No.: US 2019/0285611 A1
(43) Pub. Date: Sep. 19, 2019

(54) GENDER AND RACE IDENTIFICATION FROM BODY FLUID TRACES USING SPECTROSCOPIC ANALYSIS

(71) Applicant: The Research Foundation for the State University of New York, Albany, NY (US)

(72) Inventor: Igor K. LEDNEV, Glenmont, NY (US)

(21) Appl. No.: 15/748,769

(22) PCT Filed: Jul. 29, 2016
(86) PCT No.: PCT/US2016/044807

§ 371 Date: Jan. 30, 2018

Related U.S. Application Data
(60) Provisional application No. 62/199,079, filed on Jul. 30, 2015.

(51) Int. Cl. G01N 33/487; G01N 21/3577; G01N 21/65

(52) U.S. Cl. CPC G01N 33/487; G01N 2021/3595; G01N 21/65; G01N 21/3577

(57) ABSTRACT

The present invention relates to a method of identifying gender and/or race of a subject using a body fluid stain from the subject. This method involves providing a sample containing a body fluid stain from the subject; providing a statistical model for determination of gender and/or race of a subject; subjecting the sample or an area of the sample containing the stain to a spectroscopic analysis to produce a spectroscopic signature for the sample; and applying the spectroscopic signature for the sample to the statistical model to ascertain gender and/or race of the subject. A method of establishing a statistical model for determination of gender and/or race of a subject using a body fluid stain from the subject is also disclosed.

This application claims the benefit of U.S. Provisional Patent Application Ser. No. 62/199,079, filed Jul. 30, 2015, which is hereby incorporated by reference in its entirety.

This invention was made with government support under Award No. 2011-DN-BX-K551 awarded by the National Institute of Justice, Office of Justice Programs, U.S. Department of Justice. The government has certain rights in this invention.

### FIELD OF THE INVENTION

The present invention relates to a gender and race identification from body fluid traces using spectroscopic analysis.

### BACKGROUND OF THE INVENTION

Body fluids found at a crime scene can be some of the most valuable forms of evidence in forensic investigations. They can provide complex information about a potential suspect or victim. Therefore, a crucial step of forensic casework is the identification of biological traces such as blood, semen, saliva, or sweat (Kobilinsky, L. F., In Forensic Chemistry Handbook; John Wiley & Sons: Hoboken, N.J., pp 269-282 (2012)). Human blood is the most common body fluid found at scenes of violent crimes. Also, the amount of sample available for a forensic investigation could be extremely small. In these instances, even more care should be taken to preserve the evidence for further analysis. There are presumptive assays, such as the Kastle-Meyer test, Hemastix, Leucomalachite Green, as well as using luminol or fluorescein (Kobilinsky, L. F., In Forensic Chemistry Handbook; John Wiley & Sons: Hoboken, N.J., pp 269-282 (2012); Johnston et al., “Comparison of Presumptive Blood Test Kits Including Hexagon OBTI,” J. Forensic Sci. 53:687-689 (2008)), and confirmatory tests (microcrystal assays) for detecting and identifying of blood (Kobilinsky, L. F., In Forensic Chemistry Handbook; John Wiley & Sons: Hoboken, N.J., pp 269-282 (2012)). Nevertheless, many of these tests require the use of hazardous chemicals, and all consume part of the sample. Furthermore, the current tests can only identify the presence of blood, but do not provide investigators with any additional information about the donor. The person's race can be inferred through cranial and dental analyses (Rosas et al., “Thin-Plate Spline Analysis of the Cranial Base in African, Asian and European Populations and its Relationship with Different Malocclusions,” Arch. Oral Biol. 53:826-834 (2008); Blumenfeld, J. “Racial Identification in the Skull and Teeth,” Totem: The University of Western Ontario Journal of Anthropology 8:20-23 (2011)) and through DNA analysis (Elkins, K. M., Forensic DNA Biology: A Laboratory Manual, 1st ed.; Academic Press: Oxford, UK (2012)). Therefore, the application of a nondestructive and rapid method for reliable identification of human blood as well as providing identifiable information, such as race, would be highly advantageous in forensic casework.

Raman spectroscopy is a sensitive method for obtaining information about the chemical and biochemical composition of a sample (Skoog et al., In Principles of Instrumental Analysis, 5th ed.; Saunders College Publishing: Orlando pp 429-444 (1998)). This analytical technique is based on molecular vibrations and requires a change in polarizability. Raman spectroscopy uses monochromatic light to irradiate a sample and inelastically scatter photons, which are collected to generate a spectrum (Skoog et al., In Principles of Instrumental Analysis, 5th ed.; Saunders College Publishing: Orlando pp 429-444 (1998)). Raman spectroscopy has already been used for the analysis of various types of forensic evidence including fibers (Miller et al., “Forensic Analysis of Single Fibers by Raman Spectroscopy,” Appl. Spectrosc. 55:1729-1732 (2001)), ink (Zięba-Palus et al., “Application of the Micro-FTIR Spectroscopy, Raman Spectroscopy and XRF Method Examination of Inks,” Forensic Sci. Int. 158:164-172 (2006)), paints (Zięba-Palus et al., “Examination of Multilayer Paint Coats by the Use of Infrared, Raman and XRF Spectroscopy for Forensic Purposes,” J. Mol. Struct. 792-793:286-292 (2006)), gunshot residue (Bueno et al., “Raman Spectroscopic Analysis of Gunshot Residue Offering Great Potential for Caliber Differentiation,” Anal. Chem. 84(10):4334-9 (2012)), and bones (McLaughlin et al., “Spectroscopic Discrimination of Bone Samples from Various Species,” Am. J. Anal. Chem. 3:161-167 (2012)), to name a few. Studies on different biological traces including blood, semen, saliva, sweat, vaginal fluid, and body fluid mixtures (Virkler et al., “Raman Spectroscopy Offers Great Potential for the Nondestructive Confirmatory Identification of Body Fluids,” Forensic Sci. Int. 181(1-3):e1-e5 (2008); Virkler et al., “Raman Spectroscopic Signature of Semen and Its Potential Application to Forensic Body Fluid Identification,” Forensic Sci. Int. 193(1-3):56-62 (2009); Virkler et al., “Forensic Body Fluid Identification: the Raman Spectroscopic Signature of Saliva,” Analyst 135(3):512-7 (2010); Sikirzhytskaya et al., “Raman Spectroscopic Signature of Vaginal Fluid and Its Potential Application in Forensic Body Fluid Identification,” Forensic Sci. Int. 216(1-3):44-8 (2012); Sikirzhytski et al., “Advanced Statistical Analysis of Raman Spectroscopic Data for the Identification of Body Fluid Traces: Semen and Blood Mixtures,” Forensic Sci. Int. 222(1-3):259-265 (2012); Sikirzhytski et al., “Discriminant Analysis of Raman Spectra for Body Fluid Identification for Forensic Purposes,” Sensors 10(4):2869-2884 (2010)) were published. The interference of common substrates with the Raman signal of deposited bloodstains (McLaughlin et al., “Circumventing Substrate Interference in the Raman Spectroscopic Identification of Blood Stains,” Forensic Sci. Int. 231(1-3):157-166 (2013)) and contaminated blood traces (Sikirzhytski et al., “Forensic Identification of Blood in the Presence of Contaminations Using Raman Microspectroscopy Coupled with Advanced Statistics: Effect of Sand, Dust, and Soil,” J. Forensic Sci. 58:1141-1148 (2013)) were previously investigated. A wide study on blood traces was also conducted to understand the heterogeneous chemical composition of blood (Virkler et al., “Raman Spectroscopic Signature of Blood and its Potential Application to Forensic Body Fluid Identification,” Anal. Bioanal. Chem. 396(1):525-534 (2010)) and to distinguish between peripheral and menstrual blood (Sikirzhytskaya et al., “Raman Spectroscopy Coupled With Advanced Statistics for Differentiating Menstrual and Peripheral Blood,” J. Biophotonics 7(1-2):59-67 (2014)).

Variances in the biochemical composition of blood from donors of different races, genders, and ages have been reported by Koh et al. (Koh et al., “Comparison of Selected Blood Components by Race, Sex, and Age,”Am. J. Clin. Nutr. 33(8):1828-35 (1980)). They found a higher concentration of albumin, hemoglobin, hematocrit, serum iron, and serum triglycerides in Caucasian (CA) donors' blood than in African American (AA) donors', while AA donors had significantly higher glucose and total protein concentrations. Hemoglobin concentration has been widely studied over the last few decades (Koh et al., “Comparison of Selected Blood Components by Race, Sex, and Age,”Am. J. Clin. Nutr. 33(8):1828-35 (1980); Garn et. al.., “Lifelong Differences in Hemoglobin Levels Between Blacks and Whites,” J. Natl. Med. Assoc. 67:91-96 (1975); Johnson et al., “Advance data From Vital and Health Statistics,” The National Center for Health Statistics, U.S. Department of Health, Education, and Welfare, Public Health Service, Office of Health Research, Statistics, and Technology, 46:1-12 (1979); Meyers et al., “Components of the Difference in Hemoglobin Concentrations in Blood Between Black and White Women in the United States,” Am. J. Epidemiol. 109:539-549 (1979); Reeves et al., “Screening for Anemia in Infants: Evidence in Favor of Using Identical Hemoglobin Criteria for Blacks and Caucasians,” Am. J. Clin. Nutr. 34:2154-2157 (1981); Gam et al., “The Magnitude and the Implications of Apparent Race Differences in Hemoglobin Values,” Am. J. Clin. Nutr. 28:563-568 (1975)), and these investigations have confirmed that there is a higher amount of hemoglobin in the blood of CA subjects than AA subjects. Kramer et al. showed that CA and AA racial groups can be distinguished based on the concentration of certain enzymes (creatine kinase and lactate dehydrogenase) in blood serum (Kramer et al., “Biocatalytic Analysis of Biomarkers for Forensic Identification of Ethnicity Between Caucasian and African American Groups,”Analyst 138(21):6251-6257 (2013)). Differences between races in plasma lipids' and lipoproteins' concentrations have also been shown (Morrison et al., “Black-White Differences in Plasma Lipids and Lipoproteins in Adults: The Cincinnati Lipid Research Clinic Population Study,” Prev. Med. 8:34-39 (1979)).

The present invention is directed to overcoming these and other deficiencies in the art.

### SUMMARY OF THE INVENTION

One aspect of the present invention relates to a method of identifying gender and/or race of a subject using a body fluid stain from the subject. This method includes providing a sample containing a body fluid stain from the subject; providing a statistical model for determination of gender and/or race of a subject; subjecting the sample or an area of the sample containing the stain to a spectroscopic analysis to produce a spectroscopic signature for the sample; and applying the spectroscopic signature for the sample to the statistical model to ascertain gender and/or race of the subject.

Another aspect of the present invention relates to a method of establishing a statistical model for determination of gender and/or race of a subject using a body fluid stain from the subject. This method includes providing a plurality of samples containing a known type of body fluid stain from a subject of known race and/or gender; subjecting each sample or an area of each sample containing the stain to a spectroscopic analysis to produce a spectroscopic signature for each sample; and establishing a statistical model for determination of gender and/or race of a subject for a particular body fluid type based on said subjecting.

Due to the significant information that can be gathered from blood, it requires special attention during forensic investigations. It can even lead to identifying a suspect. All currently applied methods for collecting information about a person are destructive to the sample since they require extraction of DNA or biomarkers from a bloodstain. Treated traces can be no longer used for further examination. Finding a nondestructive method would be very valuable to support forensic investigations. Attenuated total reflectance (ATR) Fourier transform infrared (FTIR) spectroscopy was applied in order to discriminate gender and race from human blood traces. Such a person's identification is possible due to chemical and biochemical differences in blood composition from donor to donor. Advanced statistics were applied in order to enhance classification processes.

Genetic profiling (or phenotype profiling; these two terms are considered synonymous here) is a very important part of criminal investigations. Determining the suspect race and gender at the very early stages of investigation would be most important. A method for determining race and/or gender based on Raman spectra of blood, saliva, sweat, and semen samples were developed. Near-Infrared (NIR) Raman microspectroscopy and Attenuated total reflectance (ATR) Fourier transform infrared (FTIR) spectroscopy were combined with advanced statistics for developing classification models which account for the sample heterogeneity and variations with donor.

Gaining knowledge from these studies, the highly selective technique of Raman spectroscopy was applied to detect chemical and biochemical differences in dry blood traces from two different racial groups. It was already reported for different species that even if visual differentiation of Raman blood spectra is impossible advanced statistics allows for classification (Virkler et al., “Blood Species Identification for Forensic Purposes using Raman Spectroscopy Combined with Advanced Statistical Analysis,” Anal. Chem. 81(18):7773-7777 (2009); McLaughlin et al., “Discrimination of Human and Animal Blood Traces Via Raman Spectroscopy,” Forensic Sci. Int. 238(0):91-95 (2014); De Wael et al., “In Search of Blood-Detection of Minute Particles using Spectroscopic Methods,” Forensic Sci. Int. 180(1):37-42 (2008), which are hereby incorporated by reference in their entirety). Therefore, in the present application, an advanced statistical approach was utilized for discrimination processes.

The present application describes the use of genetic algorithm (GA) analysis, which helped to select the spectral regions with the largest diversity between Caucasian (CA) and African American (AA) peripheral blood donors. GA analysis is a heuristic search algorithm developed to select variables with the lowest prediction error using simulated natural processes necessary for evolution (Niazi et al., “Genetic Algorithms in Chemometrics,” Journal of Chemometrics 26(6):345-351 (2012), which is hereby incorporated by reference in its entirety). For statistical analysis, principal component analysis (PCA) was used to remove outliers (Pascoal et al., In Combining Soft Computing and Statistical Methods in Data Analysis; Borgelt et al., Eds.; Springer Berlin Heidelberg: Vol. 77:499-507 (2010), which are hereby incorporated by reference in their entirety), and support vector machine-discriminant analysis (SVM-DA) to build classification models. SVM-DA is a supervised machine learning technique that has been widely used in pattern classification problems (Sikirzhytskaya et al., “Raman Spectroscopy Coupled With Advanced Statistics for Differentiating Menstrual and Peripheral Blood,” J. Biophotonics 7(1-2):59-67 (2014); Marcelo et al., “Profiling Cocaine by ATR-FTIR,” Forensic Sci. Int. 246:65-71 (2015), which are hereby incorporated by reference in their entirety). In order to validate the accuracy performance of SVM-DA models built for this study, outer cross-validation (CV) loop was performed.

The receiver operating characteristic (ROC) and area under the curve (AUC) analyses are commonly used in diagnostic and screening tests (Hajian-Tilaki, K., “Receiver Operating Characteristic (ROC) Curve Analysis for Medical Diagnostic Test Evaluation,” Caspian J. Intern. Med. 4:627-635 (2013), which is hereby incorporated by reference in its entirety). The trapezoidal method of integration was used to estimate AUCs of ROC curves with corresponding 95% confidence intervals (CIs) that have been estimated with the method described DeLong et al., “Comparing the Areas under Two or More Correlated Receiver Operating Characteristic Curves: A Nonparametric Approach,” Biometrics 837-845 (1988), which is hereby incorporated by reference in its entirety. The curve in a ROC diagram plots sensitivity (true positive rate) against specificity (true negative rate) for varying thresholds of class prediction probabilities was generated, as a way to gauge the prediction efficiency of the SVM-DA models built. Here, a proof-of-concept that Raman spectroscopic analysis of bloodstains is able to successfully differentiate between CA and AA racial groups was demonstrated. Further studies are necessary for examining other factors and conditions, which can potentially affect the biochemical composition and corresponding Raman signature of a bloodstain.

The word “race” has become a complex and sensitive term. Some believe race to be a purely socio-cultural construct, while others report that there is biological evidence to support it (Jorde et al., “Genetic Variation, Classification, and ‘Race’,” Nature Reviews. Genetics 36(11):28-33 (2004), which is hereby incorporated by reference in its entirety). One approach has been to differentiate the two terms; “race” and “biological race” (Ousley et al., “Understanding Race and Human Variation: Why Forensic Anthropologists are Good at Identifying Race,” American Journal of Physical Anthropology 139(1):68-76 (2009), which is hereby incorporated by reference in its entirety). The first refers to the social notions about race, often characterized by broad generalizations and stereotypes. The latter refers to “a division of a species which differs from other divisions by the frequency with which certain hereditary traits appear among its members” (Brues, A. M., “People and Races,” New York: Macmillan 336 (1977), which is hereby incorporated by reference in its entirety). In this sense, “biological race” is very similar to biogeographic ancestry.

There is no technique to predict a person's race based on the Raman spectrum of a dried semen sample. In the present application, “race” refers to a self-reported characteristic that includes, but is not limited to, skin color. In the present application, it was uncritically ascribed to the hypothesis that groups from different biological races or biogeographic ancestries have biological differences, which appear be evident in skeletal morphology and genetics (Ousley et al., “Understanding Race and Human Variation: Why Forensic Anthropologists are Good at Identifying Race,” American Journal of Physical Anthropology 139(1):68-76 (2009), which is hereby incorporated by reference in its entirety). While this is absolutely a serious and important consideration, it is outside of the scope of the present work. It was hypothesized that discernible differences could be seen in the biochemical make up of semen. In the present application, Raman spectra were acquired from human semen samples, from donors of three different races (Caucasian, Black, and Hispanic). Their spectra were then analyzed and compared using MATLAB version R2012a. Statistical models were built to differentiate the spectra according to their respective races. The developed model allowed for discrimination between races with excellent sensitivity and specificity. Ultimately, all 28 donors were classified correctly. The results described show Raman spectroscopy's potential to correctly differentiate races based on dry semen traces.

In the present application Raman microspectroscopy was used for gender identification from the human blood, taking into account its heterogeneity. Advanced statistical analysis was performed to deal with variations of Raman spectra and to minimize the possibility of false gender identification. An automatic mapping technique was used to collect Raman spectra from different spots of dried blood samples. The fluorescent background was subtracted from the experimental data using an automatic baseline correction procedure, and two data sets (male and female) were formed. The present application showed that human genders could be predicted based on dry blood traces using support vector machine discriminant analysis (SVMDA) and (k-nearest neighbors) KNN algorithms with a high level of confidence. Despite the visual similarity of Raman spectra from male and female donors, the sensitivity and specificity of the SVMDA model was about 77% and 93% respectively, despite of the visual similarity of Raman spectra from male and female donors.

In the present application, ATR-FTIR spectroscopy was applied as a sensitive analytical method for human blood identification. Dissimilarities between groups of genders and races were focused on. As already reported, blood donors are ineligible for visual distinction between Raman or infrared spectra (Virkler et al., “Blood Species Identification for Forensic Purposes Using Raman Spectroscopy Combined with Advanced Statistical Analysis,” Anal. Chem. 81(18):7773-7777 (2009); McLaughlin et al., “Discrimination of Human and Animal Blood Traces Via Raman Spectroscopy,” Forensic Sci. Int. 238(0):91-95 (2014); De Wael et al., “In Search of Blood—Detection of Minute Particles Using Spectroscopic Methods,” Forensic Sci. Int. 180(1):37-42 (2008), which are hereby incorporated by reference in their entirety). In the present application supporting discrimination power was employed, with advanced statistical analysis (Wise et al., PLS_Toolbox 3.5 for Use with MATLAB Wenatchee, Wash.: Eigenvector Research, Inc. (2005), which is hereby incorporated by reference in its entirety). Firstly, genetic algorithm (GA) allowed for selection of spectral ranges where the biggest differences between the applied classes occur (Niazi et al., “Genetic Algorithms in Chemometrics,” J. Chemometrics 26(6):345-351 (2012), which is hereby incorporated by reference in its entirety). This step was carried out in two different ways: for gender discrimination and distinction between races of Caucasian (CA), African American (AA), and Hispanic (HI). A principal component analysis (PCA) model was used to remove outliers (through Q residuals and Hotelling T2) (Rodriguez et al., “Raman Spectroscopy and Chemometrics for Identification and Strain Discrimination of the Wine Spoilage Yeasts Saccharomyces cerevisiae, Zygosaccharomyces bailii, and Brettanomyces bruxellensis,” Appl. Environ. Microbiol. 79(20):6264-6270 (2013); Xiao et al., “Drift Compensation of Gas Sensor Array by Matrix Transform and Genetic Algorithm Based on Database,” J. Computational Information Systems, 9(9):3469-3476 (2013), which are hereby incorporated by reference in their entirety). Multivariate partial least squares-discriminant analysis (PLS-DA) was conducted to differentiate gender and races with emphasis on the validation phase to assure the applicability of the built models. PLS-DA is a classification method based on the standard PLS algorithm and for the dependent y-vector class labels are used (Varmuza et al., Introduction to Multivariate Statistical Analysis in Chemometrics. CRC Press (2008), which is hereby incorporated by reference in its entirety). An external cross-validation (CV) was used in order to examine prediction performance of models where all spectra from one donor were placed aside from training dataset and predicted by recalculating model based on n−1 donors. Y predictions were recorded from all donors for each spectrum and for each donor as well. Additionally, the predictive abilities of PLS-DA models were summarized using a receiver operating characteristic (ROC) and area under ROC curve (AUC). In the ROC space, the AUC is a single measure of model performance. ROC curves were generated from cross-validated Y-predicted values, and the best threshold was determined for each class prediction and for its corresponding PLS-DA classifier. The last step of validation was testing the model with external blind samples, from donors who were not included in training datasets. This approach showed potential to discriminate donors based on dry blood traces found at a crime scene. Moreover, the method gives fast results, and it is not destructive to the sample, and thus can be applied as an additional investigation technique before the sample is subjected for final DNA testing. Availability of ATR-FTIR portable instruments (Mukhopadhyay, R., “Product Review: Portable FTIR Spectrometers Get Moving,” Anal. Chem. 76(19):369 A-372 A (2004), which is hereby incorporated by reference in its entirety) raises efficacy of this approach to compare with other bloodstain tests which mostly require laboratory settings.

### BRIEF DESCRIPTION OF THE DRAWINGS

FIGS. 1A-C are graphs showing baseline corrected and normalized mean Raman spectrum of all blood samples from the training dataset with red highlighted regions showing the most significant areas for distinction between classes in dataset based on GA analysis (FIG. 1A), difference mean spectrum (black line) and the standard deviation (SD) of mean blood spectra for Raman datasets of Caucasian (blue lines) and African American (green lines) donors (FIG. 1B), and receiver operating characteristic (ROC) curves for the SVM classifiers for classification of Caucasian and African American races based on probabilities for each spectrum (upper part) and for each subject (lower part) (FIG. 1C). Area under the curve (AUC) values give the efficacies of the SVM classifiers and give the probability that the race will be classified accurately as Caucasian or African American according to Raman spectra, which is 71% based on a single spectrum and 83% based on a single donor.

FIGS. 2A-B are graphs showing mean spectra of the female (red line) and male (green line) (FIG. 2A) and standard deviation spectra calculated for female (red line) and male (green line) Raman data sets (FIG. 2B).

FIGS. 3A-B are graphs showing PCA score plots of blood spectra built using the first three principal components. FIGS. 3A and 3B show the same data observed from different points of view. Each colored symbol represents a single blood Raman spectrum acquired from samples collected from female (red triangles) and male (green crosses) donors.

FIGS. 4A-B are graphs showing Hierarchical Ward's clustering (FIG. 4A) and clusters dominated by “female” (red labels) and “male” (green labels) Raman spectra (FIG. 4B).

FIGS. 5 A-B show SVMDA analysis of Raman spectra (female—red labels, male—green labels) from two genders. FIG. 5A is a graph showing assignment of Raman spectra to female (1) and male (2) classes. FIG. 5B is a graph showing predicted probability to be assigned to female class.

FIGS. 6A-B are graphs showing an averaged Raman spectra of human blood (different colors correspond to different donors) (FIG. 6A) and SVMDA model, calculated based on averaged spectra (red triangles—female donors, green asterisks—male donors) (FIG. 6B).

FIGS. 7A-D are graphs showing spectra collected from one donor (after baseline correction), illustrating the intra-sample heterogeneity observed in semen (FIG. 7A), mean spectra of the 28 donors (after baseline correction), showing some inter-sample variation but overall consistency in major Raman peak locations (FIG. 7B), and mean spectra of Black (green), Caucasian (red), and Hispanic (blue) donors (after baseline correction) (FIGS. 7C-D).

FIG. 8 is a graph showing the cross-validated classification predictions for the 28 mean spectra, based on SVMDA model.

FIG. 9 is a graph showing the cross-validated classification predictions for all spectra, based on SVMDA model.

FIG. 10 is a graph showing the score plot for the class prediction probability obtained for individual spectra based on SVMDA model.

FIG. 11 is a scheme showing the two-step classification system for a hypothetical sample, X, with 50 spectra. The number or percentage of spectra classified as a particular race is shown in parentheses.

FIG. 12 is a scheme showing the three-step classification system for a hypothetical sample, X, with 50 spectra. The number or percentage of spectra classified as a particular race is shown in parentheses.

FIGS. 13A-B are graphs showing raw mean infrared human blood spectra of genders: male (red line), female (green line) (FIG. 13A), and races: Caucasian (red line), African American (green line), Hispanic (blue line) (FIG. 13B). The region of 1711-2669 cm⁻¹ was excluded to avoid interference from the diamond ATR crystal.

FIGS. 14A-B are graphs showing calculated receiver operating characteristic (ROC) curves using externally CV Y-prediction values of the PLS-DA models for classification of males and females for each spectrum (FIG. 14A) and for each donor (FIG. 14B). Area under the curve (AUC) refers to area under ROC curve value calculated from the model predictions against the outcome that shows the efficacies of the PLS-DA classifiers. The specificity and sensitivity are corresponding with the threshold chosen to maximize the distance to the diagonal line.

FIG. 15 is a graph showing box and whisker plots illustrating the spread of the Y predictions in external CV stratified by the class membership in gender set. The Y axis plots the probability of being predicted as male for male (red), and female (green) donors, as well as the blind tests (black, D1, D2, D3, D4). The plots show the results of predicted class labels obtained from the PLS-DA model where all spectra plotted above the threshold (dotted line) are classified as males, and those below the threshold are classified as female. The horizontal line within each box represents mean score, the boxes represent the range of values from the 10th and 90th percentile, and the ends of the whiskers represent the 5th and 95th percentile values.

FIGS. 16A-C are graphs showing calculated ROC curves using externally CV Y-prediction values of the PLS-DA models for classification of races: Caucasian (FIG. 16A), African American (FIG. 16B), Hispanic (FIG. 16C) for each spectrum (left panel) and for each donor (right panel). The specificity and sensitivity are corresponding with the threshold chosen to maximize the distance to the diagonal line.

FIGS. 17A-C are graphs showing box and whisker plots illustrating the spread of the Y predictions in external CV stratified by the class membership in race set for Caucasian (red) (FIG. 17A), (b) African American (green) (FIG. 17B), and Hispanic (blue) (FIG. 17C) PLS-DA models. The black boxes represent predictions of corresponding race in blind test divided into a single donor (D1, D2, D3, D4). The plots show results of predicted class label obtained using PLS-DA models where all spectra being classified as corresponding race (above dotted threshold line) or not (below threshold).

FIG. 18 is a graph showing the background spectrum of the ATR crystal of instrument.

FIGS. 19A-B are graphs showing pretreated infrared spectra with selected regions for distinction between classes of males and females (FIG. 19A) and Caucasian, Black, and Hispanic donors (FIG. 19B). Genetic algorithm (GA) analysis was applied to assess variables giving the strongest discrimination power for genders and races. The region of 1711-2669 cm⁻¹ was excluded due to interference from the ATR crystal (with peaks not corresponding to vibrations of blood molecules).

FIGS. 20A-B are graphs showing an average normalized Raman spectra from saliva traces. Spectra are colored according to donor (FIG. 20A) and race (FIG. 20B).

FIG. 21 is a graph showing a cross-validated class prediction score plot from the SVM-DA model to differentiate Raman spectra of Caucasian (red diamonds), Black (green squares), and Asian (cyan triangles) saliva donors. Each data point represents a single Raman spectrum.

FIG. 22 is a graph showing an average normalized Raman spectra of female (red) and male (green) donors.

FIGS. 23A-B are graphs showing results from the SVM-DA model to differentiate Raman spectra from female (red diamonds) and male (green squares) saliva donors. Each data point represents a single Raman spectrum. FIG. 23 A shows cross-validated class prediction score plot. FIG. 23B shows class prediction probability plot, with the y-axis plotting the probability of a spectrum being assigned to the male class.

FIG. 24 is a graph showing mean preprocessed Raman spectra from all 20 sweat donors.

FIG. 25 is a graph showing mean preprocessed Raman spectra from Caucasian (red), Black (green), Hispanic (royal blue), and Asian (cyan) sweat donors.

FIG. 26 is a scores plot from the SVM-DA model showing the most probable racial class predictions for the calibration dataset of sweat spectra. Each symbol on the scores plot represents a single spectrum from a Caucasian (red diamond), Black (green square), Hispanic (royal blue triangle), or Asian (cyan triangle) donor.

FIG. 27 is a graph showing mean preprocessed Raman spectra of female (red) and male (green) sweat donors.

FIG. 28 is a scores plot from the SVM-DA model showing the most probable gender class predictions for the calibration dataset of sweat spectra. Each symbol on the scores plot represents a single spectrum from a female (red diamond) or male (green square) donor.

FIGS. 29A-C are graphs showing mean Raman spectra of semen obtained for Caucasian (FIG. 29A), Black (FIG. 29B), and Hispanic (FIG. 29C) donors. Mean spectra (red lines) and spectral variations around the mean+/−2 STD (black areas) are shown.

FIGS. 30 A-C are graphs showing preprocessed Raman spectra of menstrual blood collected from all 15 donors (FIG. 30A), averaged by donor (FIG. 30B), and averaged by race (FIG. 30C).

FIG. 31 is an averaged preprocessed menstrual blood spectra showing peaks selected by genetic algorithm analysis in a darker shade of red (African American) and green (Caucasian).

FIG. 32 is a graph showing cross-validated results for African American class predictions for the second PLS-DA model (built with GA selected peaks).

FIG. 33 is a graph showing scores plot showing class prediction probability as African American for the first SVM-DA model built with 225 spectra.

FIG. 34 is a graph showing results for class prediction probability as African American for the second SVM-DA model built with 225 spectra.

FIGS. 35A-B are graphs showing SVM-DA calibration model of race (red—Caucasian, green—black, blue—Hispanic) (FIG. 35A) and gender (red—male, green—female) (FIG. 35B) differentiation based on individual spectra.

FIGS. 36A-C are graphs showing ROC curves for the SVM classifiers for classification of Caucasian (FIG. 36A), Hispanic (FIG. 36B), and Black (FIG. 36C) races based on probabilities for each spectrum. The dots indicate the value corresponding to a threshold while the numbers in parentheses correspond to specificity and sensitivity.

FIGS. 37A-C are graphs showing ROC curves for the SVM classifiers for classification of Caucasian (FIG. 37A), Hispanic (FIG. 37B), and Black (FIG. 37C) races based on probabilities for each subject. The dots indicate the value corresponding to a threshold while the numbers in parentheses correspond to specificity and sensitivity.

FIGS. 38A-B are graphs showing ROC curves for the SVM classifier for classification of males and females based on probabilities for each spectrum (FIG. 38A) and for each subject (FIG. 38B). The dots indicate the value corresponding to a threshold while the numbers in parentheses correspond to specificity and sensitivity.

### DETAILED DESCRIPTION OF THE INVENTION

One aspect of the present invention relates to a method of identifying gender and/or race of a subject using a body fluid stain from the subject. This method includes providing a sample containing a body fluid stain from the subject; providing a statistical model for determination of gender and/or race of a subject; subjecting the sample or an area of the sample containing the stain to a spectroscopic analysis to produce a spectroscopic signature for the sample; and applying the spectroscopic signature for the sample to the statistical model to ascertain gender and/or race of the subject.

In one embodiment, the body fluid is selected from the group consisting of blood, saliva, sweat, urine, semen, and vaginal fluid. In a preferred embodiment, the body fluid is blood.

In one embodiment, the gender of the subject is determined.

In another embodiment, the race of the subject is determined.

In one embodiment, the method determines the race of the subject as being black, white, asian, or hispanic.

In one embodiment, the sample is recovered at a crime scene.

In another embodiment, spectroscopic analysis is selected from the group consisting of Raman spectroscopy, mass spectrometry, fluorescence spectroscopy, laser induced breakdown spectroscopy, infrared spectroscopy, scanning electron microscopy, X-ray diffraction spectroscopy, powder diffraction spectroscopy, X-ray luminescence spectroscopy, inductively coupled plasma mass spectrometry, capillary electrophoresis, and atomic absorption spectroscopy.

Raman spectroscopy is a spectroscopic technique which relies on inelastic or Raman scattering of monochromatic light to study vibrational, rotational, and other low-frequency modes in a system (Gardiner, D. J., Practical Raman Spectroscopy, Berlin: Springer-Verlag, pp. 1-3 (1989), which is hereby incorporated by reference in its entirety). Vibrational modes are very important and very specific for chemical bonds in molecules. They provide a fingerprint by which a molecule can be identified. The Raman effect is obtained when a photon interacts with the electron cloud of a molecular bond exciting the electrons into a virtual state. The scattered photon is shifted to lower frequencies (Stokes process) or higher frequencies (anti-Stokes process) as it abstracts or releases energy from the molecule. The polarizability change in the molecule will determine the Raman scattering intensity, while the Raman shift will be equal to the vibrational intensity involved.

Raman spectroscopy is based upon the inelastic scattering of photons or the Raman shift (change in energy) caused by molecules. The analyte is excited by laser light and upon relaxation scatters radiation at a different frequency which is collected and measured. With the availability of portable Raman spectrometers, it is possible to collect Raman spectra in the field. Using portable Raman spectrometers offers distinct advantages to government agencies, first responders, and forensic scientists (Hargreaves et al., “Analysis of Seized Drugs Using Portable Raman Spectroscopy in an Airport Environment—a Proof of Principle Study,” J. Raman Spectroscopy 39(7):873-880 (2008), which is hereby incorporated by reference in its entirety).

Raman spectroscopy is increasing in popularity among the different disciplines of forensic science. Some examples of its use today involve the identification of drugs (Hodges et al., “The Use of Fourier Transform Raman Spectroscopy in the Forensic Identification of Illicit Drugs and Explosives,”Molecular Spectroscopy 46:303-307 (1990), which is hereby incorporated by reference in its entirety), lipsticks (Rodger et al., “The In-Situ Analysis of Lipsticks by Surface Enhanced Resonance Raman Scattering,” Analyst 1823-1826 (1998), which is hereby incorporated by reference in its entirety), and fibers (Thomas et al., “Raman Spectroscopy and the Forensic Analysis of Black/Grey and Blue Cotton Fibers Part 1: Investigation of the Effects of Varying Laser Wavelength,” Forensic Sci. Int. 152:189-197 (2005), which is hereby incorporated by reference in its entirety), as well as paint (Suzuki et al., “In Situ Identification and Analysis of Automotive Paint Pigments Using Line Segment Excitation Raman Spectroscopy: I. Inorganic Topcoat Pigments,” J. Forensic Sci. 46:1053-1069 (2001), which is hereby incorporated by reference in its entirety) and ink (Mazzella et al., “Raman Spectroscopy of Blue Gel Pen Inks,” Forensic Sci. Int. 152:241-247 (2005), which is hereby incorporated by reference in its entirety) analysis. Very little or no sample preparation is needed, and the required amount of tested material could be as low as several picograms or femtoliters (10⁻¹² gram or 10⁻¹⁵ liter, respectively). A typical Raman spectrum consists of several narrow bands and provides a unique vibrational signature of the material (Grasselli et al., “Chemical Applications of Raman Spectroscopy,” New York: John Wiley & Sons (1981), which is hereby incorporated by reference in its entirety). Unlike infrared (IR) absorption spectroscopy, another type of vibrational spectroscopy, Raman spectroscopy shows very little interference from water (Grasselli et al., “Chemical Applications of Raman Spectroscopy,” New York: John Wiley & Sons (1981), which is hereby incorporated by reference in its entirety). Proper Raman spectroscopic measurements do not damage the sample. A swab could be tested in the field and still be available for further use in the lab, and that is very important to forensic application. The design of a portable Raman spectrometer is a reality now (Yan et al., “Surface-Enhanced Raman Scattering Detection of Chemical and Biological Agents Using a Portable Raman Integrated Tunable Sensor,” Sensors and Actuators B. 6 (2007); Eckenrode et al., “Portable Raman Spectroscopy Systems for Field Analysis,” Forensic Science Communications 3:(2001), which are hereby incorporated by reference in their entirety) which could lead to the ability to make identifications at the crime scene.

Fluorescence interference is the largest problem with Raman spectroscopy and is perhaps the reason why the latter technique has not been more popular in the past. If a sample contains molecules that fluoresce, the broad and much more intense fluorescence peak will mask the sharp Raman peaks of the sample. There are a few remedies to this problem. One solution is to use deep ultraviolet (DUV) light for exciting Raman scattering (Lednev I. K., “Vibrational Spectroscopy: Biological Applications of Ultraviolet Raman Spectroscopy,” in: V. N. Uversky, and E. A. Permyakov, Protein Structures, Methods in Protein Structures and Stability Analysis (2007), which is hereby incorporated by reference in its entirety). Practically no condensed face exhibits fluorescence below ˜250 nm. Possible photodegradation of biological samples is an expected disadvantage of DUV Raman spectroscopy. Another option to eliminate fluorescence interference is to use a near-IR (NIR) excitation for Raman spectroscopic measurement. Finally, surface enhanced Raman spectroscopy (SERS) which involves a rough metal surface can also alleviate the problem of fluorescence (Thomas et al., “Raman Spectroscopy and the Forensic Analysis of Black/Grey and Blue Cotton Fibers Part 1: Investigation of the Effects of Varying Laser Wavelength,” Forensic Sci. Int. 152:189-197 (2005), which is hereby incorporated by reference in its entirety). However, this method requires direct contact with the analyte and cannot be considered to be nondestructive.

Basic components of a Raman spectrometer are (i) an excitation source; (ii) optics for sample illumination; (iii) a single, double, or triple monochromator; and (iv) a signal processing system consisting of a detector, an amplifier, and an output device.

Typically, a sample is exposed to a monochromatic source usually a laser in the visible, near infrared, or near ultraviolet range. The scattered light is collected using a lens and is focused at the entrance slit of a monochromator. The monochromator which is set for a desirable spectral resolution rejects the stray light in addition to dispersing incoming radiation. The light leaving the exit slit of the monochromator is collected and focused on a detector (such as a photodiode arrays (PDA), a photomultiplier (PMT), or charge-coupled device (CCD)). This optical signal is converted to an electrical signal within the detector. The incident signal is stored in computer memory for each predetermined frequency interval. A plot of the signal intensity as a function of its frequency difference (usually in units of wavenumbers, cm⁻¹) will constitute the Raman spectroscopic signature.

Raman signatures are sharp and narrow peaks observed on a Raman spectrum. These peaks are located on both sides of the excitation laser line (Stoke and anti-Stoke lines). Generally, only the Stokes region is used for comparison (the anti-Stoke region is identical in pattern, but much less intense) with a Raman spectrum of a known sample. A visual comparison of these set of peaks (spectroscopic signatures) between experimental and known samples is needed to verify the reproducibility of the data. Therefore, establishing correlations between experimental and known data is required to assign the peaks in the molecules, and identify a specific component in the sample.

The types of Raman spectroscopy suitable for use in conjunction with the present invention include, but are not limited to, conventional Raman spectroscopy, Raman microspectroscopy, near-field Raman spectroscopy, including but not limited to the tip-enhanced Raman spectroscopy, surface enhanced Raman spectroscopy (SERS), surface enhanced resonance Raman spectroscopy (SERRS), and coherent anti-Stokes Raman spectroscopy (CARS). Also, both Stokes and anti-Stokes Raman spectroscopy could be used.

In addition to Raman spectroscopy, the spectroscopic analysis of the present invention can be performed using, for example, mass spectrometry, fluorescence spectroscopy, laser induced breakdown spectroscopy, infrared spectroscopy, scanning electron microscopy, X-ray diffraction spectroscopy, powder diffraction spectroscopy, X-ray luminescence spectroscopy, inductively coupled plasma mass spectrometry, capillary electrophoresis, or atomic absorption spectroscopy. Some of the spectroscopic methods mentioned above, including but not limited to Raman spectroscopy, are relatively simple, rapid, non-destructive, and would allow for the development of a portable instrument. The technique can be performed with relatively small samples, picogram (pg) quantities. The composition of the sample is not changed in any way, allowing for further forensic tests on the residue or other components of the evidence.

Scanning Electron Microscopy combined with Energy Dispersive Spectroscopy (SEM/EDS or EDX when equipped with an X-ray analyzer) is capable of obtaining both morphological information and the elemental composition. Recently, SEM/EDS systems have become automated, making automated computer-controlled SEM the method of choice for most laboratories conducting analyses. Several features of the SEM make it useful in many forensic studies, including magnification, imaging, composition analysis, and automation.

Inductively coupled plasma mass spectrometry (ICP-MS) is a mass analysis method with sensitivity to metals. As a result, this analytical technique is ideal for analyzing barium, lead, and antimony. This technique is known for its sensitivity, having detection limits that are usually in the parts per billion.

Fourier transform infrared (FTIR) spectroscopy is a versatile tool for the detection, estimation and structural determination of organic compounds such as drugs, explosives, and organic components. Due to the availability of portable IR spectrometers, it will be possible to analyze the samples at scenes remote from laboratories. Capillary electrophoresis (CE) is another suitable analytical technique. The significant advantage of CE is the low probability of false positives (Bell, S., Forensic Chemistry, Pearson Education: Upper Saddle River, N.J. (2006), which is hereby incorporated by reference in its entirety).

Atomic absorption spectroscopy (AAS) is a bulk method of analysis used in the analysis of inorganic materials in primer residue, namely Ba and Sb. The high sensitivity for a small volume of sample is one advantage of AAS. This technique involves the absorption of thermal energy by the sample and subsequent emission of some or all of the energy in the form of radiation (Bauer et al., Instrumental Analysis, Allyn and Bacon, Inc.: Boston (1978), which is hereby incorporated by reference in its entirety). These emissions are generally unique for specific elements and thus give information about the composition of the sample. Laser-induced breakdown spectroscopy (LIBS) is a type of atomic emission spectroscopy that implements lasers to excite the sample. Rather than flame AAS, LIBS is accessible to field testing because of the availability of portable LIBS systems.

X-ray diffraction (XRD) is one such technique that can be used for the characterization of a wide variety of substances of forensic interest (Abraham et al., “Application of X-Ray Diffraction Techniques in Forensic Science,” Forensic Science Communications 9(2) (2007), which is hereby incorporated by reference in its entirety). XRD is capable of obtaining information about the actual structure of samples, in a non-destructive manor.

In one embodiment, spectroscopic analysis is Raman spectroscopy. In a preferred embodiment, Raman spectroscopy is selected from the group consisting of resonance Raman spectroscopy, normal Raman spectroscopy, Raman microscopy, Raman microspectroscopy, NIR Raman spectroscopy, surface enhanced Raman spectroscopy (SERS), tip enhanced Raman spectroscopy (TERS), Coherent anti-Stokes Raman scattering (CARS), and Coherent anti-Stokes Raman scattering microscopy.

In another embodiment, spectroscopic analysis is Infrared spectroscopy. In a preferred embodiment, the Infrared spectroscopy is selected from the group consisting of Infrared microscopy, Infrared microspectroscopy, Infrared reflection spectroscopy, Infrared absorption spectroscopy, attenuated total reflection infrared spectroscopy, Fourier transform infrared spectroscopy, and attenuated total reflection Fourier transform infrared spectroscopy.

The spectroscopic signature can be obtained from: spectra at different locations of the sample of the body fluid; a single spectrum of the sample of the body fluid; or as an average of spectra collected at different locations of the sample.

In the present invention, the term “spectroscopic signature” refers to a single spectrum, an averaged spectrum, multiple spectra, or any other spectroscopic representation of intrinsically heterogeneous samples.

In one embodiment, the statistical model for determination of gender and/or race of a subject is prepared by multivariate analysis. In a preferred embodiment, multivariate analysis is supervised multivariate analysis.

In another embodiment, the statistical model is prepared by classification statistical analysis. In a preferred embodiment, the classification statistical analysis is selected from the group consisting of Partial least squares discriminant analysis (PLS-DA), Support vector machines discriminant analysis (SVMDA), K-Nearest neighbor (KNN), Artificial neural network (ANN), and Soft independent modeling of/by class analogy (SIMCA).

Artificial neural network (ANN) are a family of models inspired by biological neural networks (the central nervous systems of animals, in particular the brain) which are used to estimate or approximate functions that can depend on a large number of inputs and are generally unknown. Artificial neural networks are typically specified using architecture, activity rule, and learning rule.

Classical least squares (CLS) techniques also known as direct least squares or forward least squares. CLS methods are typically used for exploratory analysis, detection, classification, and quantification. CLS regression methods include classical, extended, weighted, and generalized least squares. These methods can be used to account for interferents (i.e. analytes other than the one of interest) in spectroscopic systems. CLS also provides a natural framework for the development of popular de-cluttering methods such as External Parameter Orthogonalization (EPO) and Generalized Least Squares (GLS) weighting.

Locally weighted regression (LWR) is a memory-based method that performs a regression around a point of interest using only training data that are “local” to that point.

Multiple linear regression (MLR) is the most common form of linear regression analysis. As a predictive analysis, the multiple linear regression is used to explain the relationship between one continuous dependent variable from two or more independent variables. The independent variables can be continuous or categorical.

Multiway partial least squares (MPLS) is an extension of the ordinary regression model PLS to the multi-way case. In chemometrics, there is some confusion in distinguishing between multi-way methods and multi-way data. Bilinear two-way PLS and PCA can cope with multi-way data by unfolding the data arrays to matrices, but the methods themselves are not multi-way and do not take advantage of any multi-way structure in the data.

Principle component regression (PCR) is a regression analysis technique that is based on principal component analysis (PCA). It considers regressing the outcome (also known as the response or, the dependent variable) on a set of covariates (also known as predictors or, explanatory variables or, independent variables) based on a standard linear regression model, but uses PCA for estimating the unknown regression coefficients in the model.

Support vector machines (SVM) are supervised learning models with associated learning algorithms that analyze data used for classification and regression analysis. Given a set of training examples, each marked for belonging to one of two categories, an SVM training algorithm builds a model that assigns new examples into one category or the other, making it a non-probabilistic binary linear classifier.

Partial least squares (PLS) or Partial least squares regression (PLSR) is a statistical method that bears some relation to principal components regression; instead of finding hyperplanes of minimum variance between the response and independent variables, it finds a linear regression model by projecting the predicted variables and the observable variables to a new space. Because both the X and Y data are projected to new spaces, the PLS family of methods are known as bilinear factor models. Partial least squares Discriminant Analysis (PLS-DA) is a variant used when the Y is categorical.

Linear discriminant analysis (LDA) is a generalization of Fisher's linear discriminant, a method used in statistics, pattern recognition and machine learning to find a linear combination of features that characterizes or separates two or more classes of objects or events.

Multivariate analysis of variance (MANOVA) is a procedure for comparing multivariate sample means. As a multivariate procedure, it is used when there are two or more dependent variables, and is typically followed by significance tests involving individual dependent variables separately.

K-Nearest neighbor (KNN) is a non-parametric method used for classification and regression. In both cases, the input consists of the k closest training examples in the feature space.

Soft independent modeling of/by class analogy (SIMCA) is a statistical method for supervised classification of data. The method requires a training data set consisting of samples (or objects) with a set of attributes and their class membership. The term soft refers to the fact the classifier can identify samples as belonging to multiple classes and not necessarily producing a classification of samples into non-overlapping classes.

Another aspect of the present invention relates to a method of establishing a statistical model for determination of gender and/or race of a subject using a body fluid stain from the subject. This method includes providing a plurality of samples containing a known type of body fluid stain from a subject of known race and/or gender; subjecting each sample or an area of each sample containing the stain to a spectroscopic analysis to produce a spectroscopic signature for each sample; and establishing a statistical model for determination of gender and/or race of a subject for a particular body fluid type based on said subjecting.

For samples containing a known type of body fluid stain from a subject of known race and/or gender the spectroscopic signature is obtained from the spectra at: different locations of the same sample of the body fluid; different samples of the same type of body fluid; or different locations on different samples of the same type of body fluid.

According to the present invention, a statistical model for determination of gender and/or race of a subject using a body fluid stain from the subject can be prepared using any type of the statistical analysis described above.

In one embodiment, the statistical model for determination of gender and/or race of a subject is prepared by multivariate analysis. In a preferred embodiment, multivariate analysis is supervised multivariate analysis.

In another embodiment, the statistical model is prepared by classification statistical analysis. In a preferred embodiment, the classification statistical analysis is selected from the group consisting of Partial least squares discriminant analysis (PLS-DA), Support vector machines discriminant analysis (SVMDA), K-Nearest neighbor (KNN), Artificial neural network (ANN), and Soft independent modeling of/by class analogy (SIMCA).

In another embodiment, the method further includes rebuilding the statistical model; and validating the statistical model.

In yet another embodiment, the method further includes performing an informative spectral features selection for further developing a spectroscopic signature.

In one embodiment, the establishing produces a statistical model for determination of the subject's gender for a specific type of body fluid.

In another embodiment, the establishing produces a statistical model for determination of the subject's race for a specific type of body fluid.

According to one embodiment, the method of developing a statistical model for determination of gender and/or race of a subject using a body fluid stain from the subject using spectroscopic analysis involves the following steps. First, multiple spectra for samples of body fluid of known gender and race are collected. Second, these spectra are preprocessed. The preprocessing step can be performed using any of the different pre-treatment procedures alone or in different combinations. Then a statistical model is developed using any of the statistical methods described above alone or in combination. Next, an informative spectral features selection is performed. Next, the model is rebuilt and, if necessary, the model can be validated using any of the statistical methods described above alone or in combination (validation step is optional).

According to another embodiment, the method of determining gender and/or race of an unknown sample involves the following steps. First, multiple spectra for an unknown sample are obtained. Second, spectra are preprocessed. Preprocessing step can be performed using any of the above-described pre-treatment procedure alone or in different combinations. Next, the statistical model for determining gender and/or race of a subject is applied to determine the gender and/or race of a subject using a body fluid stain.

### EXAMPLES

### Example 1—Sample Preparation for Examples 2-4

A total of 20 human peripheral blood samples were used for this experiment, which were purchased from Bioreclamation, Inc. Donors were chosen with consideration to gender and age diversity. The average age of Caucasian (CA) and African American (AA) donors was 45.0±8.4 and 43.8±7.2 years, respectively, with male donors making up 40% and 50% of the donor pool, respectively. All blood samples were kept frozen until sample preparation. After defrosting, tubes of blood were vortexed and 10 μL of blood were deposited onto an aluminum foil covered microscope slide. Prepared samples were allowed to dry overnight prior to spectral collection.

### Example 2—Instrumentation and Spectral Collection

A Renishaw inVia Raman spectrometer was used for sample analysis. The instrument was equipped with a Leica optical microscope with a 20× objective and PRIOR automatic stage. A 785 nm laser light (power=4.0 mW) was used for excitation; twenty 10-second accumulations were recorded from each spot on the sample. Spectra were recorded in the range of 250-1800 cm⁻¹. A total of 180 spectra were collected using Raman mapping with nine different spots for each sample. The instrument was calibrated using a silicon standard (peak at 520.6 cm⁻¹) before collecting spectra from a bloodstain.

### Example 3—Data Treatment and Validation

Data treatment and advanced statistical analysis were performed using MATLAB R2013b (Mathworks, Inc.). Recorded blood spectra were divided into two datasets based on race. Raman spectra were baseline corrected using the automatic weighted least squares baseline algorithm, normalized by the standard normal variate method, and mean centered. After these preprocessing steps, further analysis was performed using the PLS Toolbox (Eigenvector Research, Inc.). Informative spectral regions were identified using genetic algorithm (GA) analysis. Multivariate outlier removal was carried out using PCA prior to all statistical analyses, which resulted in the removal of 20 spectra from the 180 total spectra originally collected. To distinguish between blood spectra from CA and AA donors, SVM-DA models were built. The method was validated by outer subject-wise CV loop where all spectra from one donor were taken out, one at a time, from the training dataset and used for validation. The remaining spectra of n−1 donors were used as training data to build a new SVM-DA model and predictions were performed for the validation data (excluded donor's spectra). For evaluation purposes, receiver operating characteristic (ROC) and area under the curve (AUC) analyses were applied. ROC analysis was carried out with the open source package pROC (Robin et al., “pROC: An Open-Source Package for R and S+ to Analyze and Compare ROC Curves.” BMC Bioinformatics 12(1):77 (2011), which is hereby incorporated by reference in its entirety). The AUC analysis indicated how well the model ranks subjects according to the probability of assignment to the correct class.

### Example 4—Results and Discussion of Examples 1-3

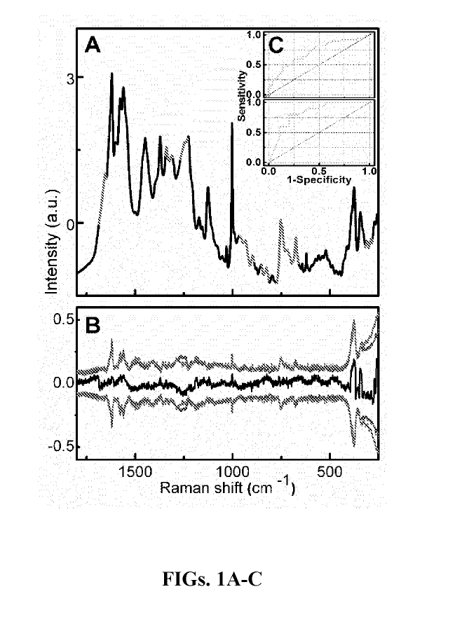

As previously mentioned, other studies have shown that visual distinction between Raman spectra of blood from different classes is not possible (Virkler et al., “Blood Species Identification for Forensic Purposes using Raman Spectroscopy Combined with Advanced Statistical Analysis,” Anal. Chem. 81(18):7773-7777 (2009); McLaughlin et al., “Discrimination of Human and Animal Blood Traces Via Raman Spectroscopy,” Forensic Sci. Int. 238(0):91-95 (2014); De Wael et al., “In Search of Blood-Detection of Minute Particles using Spectroscopic Methods,” Forensic Sci. Int. 180(1):37-42 (2008), which are hereby incorporated by reference in their entirety). This is due to the fact that spectra generated by Raman analysis of dried blood, using 785 nm excitation, are composed of peaks originating exclusively from vibrational modes of hemoglobin, which is present in all human blood samples (Premasiri et al., “Surface-Enhanced Raman Scattering of Whole Human Blood, Blood Plasma, and Red Blood Cells: Cellular Processes and Bioanalytical Sensing,” J. Phys. Chem. B, 116(31):9376-86 (2012), which is hereby incorporated by reference in its entirety). The averaged preprocessed spectrum of all CA and AA donors analyzed in this study is shown in FIG. 1A. It was not surprising that Raman spectra for both classes were similar since human blood consists of the same components, with only quantitative variations between them for different races. The number of peaks for both races was equivalent and no spectral shifts were evident. However, some slight intensity variations were detected in the regions 250-400 cm⁻¹ and 1230-1268 cm⁻¹, which were also illustrated by the difference spectrum for these two classes (FIG. 1B). Additionally, visual differences in peak intensities appeared at 1000 cm⁻¹ (phenylalanine), 1575 cm⁻¹(proteins), and 1620 cm⁻¹(heme) (Sikirzhytskaya et al., “Raman Spectroscopy Coupled With Advanced Statistics for Differentiating Menstrual and Peripheral Blood,” J. Biophotonics 7(1-2):59-67 (2014)), which is hereby incorporated by reference in its entirety). This slightly higher intensity of heme for CA donors is supported by a previous study which showed higher hemoglobin concentration for the CA race in comparison to the AA race (Koh et al., “Comparison of Selected Blood Components by Race, Sex, and Age,” Am. J. Clin. Nutr. 33(8):1828-35 (1980), which is hereby incorporated by reference in its entirety). The average difference (FIG. 1B, black line) between Raman spectra in CA and AA datasets is smaller than one standard deviation between individual spectra in each dataset (blue and green lines). This limits the opportunity to use the appearance of individual bands in a Raman spectrum for race identification and indicates the need for advanced statistical analysis using the entire spectral range.

GA analysis was carried out on the 160 spectra used to build the SVM-DA models for optimization purposes and to better understand and identify the origin of differences between classes. The analysis considered all possible variables (wavenumbers) within the Raman spectral dataset and their significance for the discrimination between classes (races). This allowed for the reduction of the original Raman spectra to subsets of unique wavenumbers in order to achieve better prediction performance (Niazi et al., “Genetic Algorithms in Chemometrics,” Journal of Chemometrics 26(6):345-351 (2012), which is hereby incorporated by reference in its entirety). The GA analysis only selected variables that gave the most valuable information for discrimination within the entire training dataset of donors from both races. The spectral regions selected by the GA operation are shown in FIG. 1A. The two regions 281-318 cm^{-l} and 1231-1268 cm⁻¹ (selected by GA analysis) are included in those that were observed to vary in intensity by visual comparison as shown in the difference spectrum (FIG. 1B).

An SVM-DA classification model was built based on 160 spectra from 20 donors (10 for each race). The model was used to differentiate races based on the spectral features, selected by GA analysis from the original Raman spectra. The SVM-DA model was automatically trained with a dataset of labeled spectra and by tuning parameters via modification of the underlying kernel function. For this study, pattern recognition SVM-DA was used with the radial basis function as a kernel function, and it was optimized by a combined approach of 5-fold CV and a systematic grid search of the parameters. The internal CV executed by the model showed 71% accuracy. The prediction performance of the subsequently built SVM-DA models was estimated by the outer loop of leave-one-out CV at the donor level. For additional information, see Varma et al., “Bias in Error Estimation When Using Cross-Validation for Model Selection,” BMC Bioinformatics 7:91 (2006), which is hereby incorporated by reference in its entirety.

All spectra from one subject at a time were excluded from the initial training set and used as the validation set to test the model built using spectra from the remaining (n−1) donors. This process was repeated until all subjects were separately used for validation.

For each donor, the final classification results were calculated as prediction probabilities that each spectrum will be correctly classified and also that each subject belongs to the correct class based on the classification of all donors' spectra. For each donor, the final classification results were calculated as prediction probabilities that each spectrum, or each subject as a whole, belong to the correct class. Among the subsets from all 20 subjects, the predicted group membership and probabilities, for each spectrum and for each subject, were recorded. Using ROC analysis, the best thresholds were identified (above which the spectrum/donor probability estimate was assigned to the correct class) to rank the SVM classifier's ability to separate the races. The results of the AUC analysis can range from 0 to 1. An AUC value of 0.5 represents a random classifier and an AUC value of 1.0 indicates a perfect test. This analysis allowed for discrimination of CA and AA races with an AUC value of 0.71 (95% CI: 0.63-0.79) based on a single spectrum, and 0.83 (95% CI: 0.64-1.00) based on each subject (FIG. 1C). These values represent the probability that the classifier can correctly distinguish between the CA and AA blood samples. The discriminatory power of the SVM-DA model was lower for a single spectrum as compared to the subject-wise results. This can be explained due to the fact that not all spectra have noticeable contributions from biomarkers with high discriminatory power.

This preliminary study showed promise for race differentiation based on human blood traces analyzed by Raman spectroscopy.

### Conclusions

For the first time, Raman spectroscopy, combined with chemometrics, has been used to differentiate between dry blood traces from CA and AA donors. To validate the internal CV results, which achieved 71% correct classification of donors based on all spectra included in a training dataset, outer CV was performed. The summary of predictions from outer CV for 20 different SVM-DA models demonstrated 83% (AUC) probability of correct race classification of individual donors after ROC analysis. These results showed promise for discrimination of the race of human peripheral blood found at a crime scene. Since blood composition quantitatively varies for different races, these changes for the two races considered here may be detected by Raman spectroscopy. More importantly, chemometrics was applied to support and strengthen the classification. This approach allowed for nondestructive detection of minor differences that were present in blood spectra between two races (CA and AA).

By using Raman spectroscopy for the method of analysis, the bloodstain's integrity was preserved, and it can be further examined or used for subsequent tests (e.g. DNA profiling) with no change to the sample. Therefore, this technique could extract information about an unknown blood sample without damaging or consuming it, unlike most tests currently used for blood identification and/or analysis in forensic casework. The application of Raman spectroscopy in real crime scene investigations is highly probable due to commercially available portable instruments, which allow for nondestructive and rapid examination at the scene of a crime. Furthermore, not only can a stain be identified as blood using the present technology but, by incorporating statistical analysis, more information about the donor can be obtained, all in a reliable and statistically confident manner.

### Example 5—Materials and Methods for Example 6

Samples

A total 30 male and 30 female blood samples, purchased from certified company Bioreclamation Inc., were used for the entire study. All donors were found to be negative for HIV ½ AB and HCV AB and non-reactive for HBSAG, HIV-1 RNA, HCV RNA, and STS. The average age for all subjects was 42 years. Samples were prepared by putting a 10-μl drop on aluminum foil placed on microscopic slide. Aluminum foil has a low level of fluorescence and very weak Raman signal. It is also an inexpensive material, which can be easily prepared right before an experiment. A Raman mapping procedure was performed on dry spots with one 10-second accumulation of 785-nm laser light with approximately 10 mW power of excitation beam. Total more than 4,500 spectra were collected from the area about 4×4 mm using a PRIOR automatic stage, attached to a Renishaw inVia confocal Raman spectrometer equipped with a research-grade Leica microscope with a 50× long-range objective (numerical aperture of 0.35). A silicon standard was used for the calibration.

Data Treatment

The spectra were imported into MATLAB 7.11 for statistical analysis. The fluorescent background contribution in Raman spectra of blood was removed using an adaptive iteratively reweighted penalized least squares (air-PLS) baseline correction algorithm. No contribution of aluminum substrate was found in the Raman spectra of blood. All Raman spectra were subjected to the statistical analysis including significant factor analysis (SFA), principal component analysis (PCA), hierarchical clustering such as k-nearest neighbor (KNN), and support vector machine discriminant analysis (SVMDA).

### Example 6—Results and Discussion of Example 5

Raman Spectra of Blood

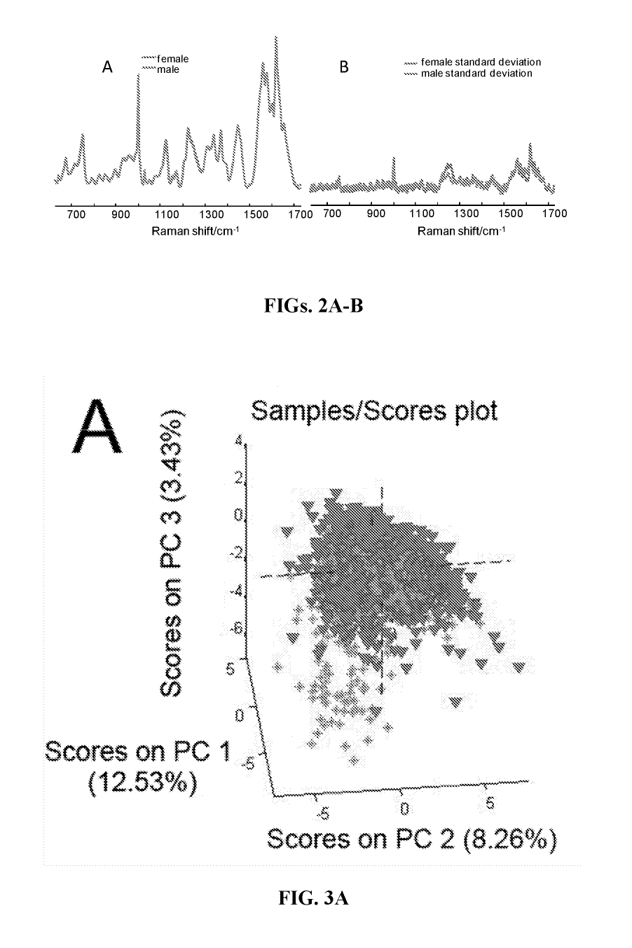

Human blood consists of a diverse biochemical constituents and their contribution varies from donor to donor (Virkler et al., “Raman Spectroscopic Signature of Blood and Its Potential Application to Forensic Body Fluid Identification,” Anal. Bioanal. Chem. 396(1): 525-34 (2010), which is hereby incorporated by reference in its entirety). The heterogeneous nature of such system could be illustrated by deviations in Raman spectra (FIGS. 2A-B). The main components of blood are red and white blood cells, thrombocytes, and biomolecules such as hemoglobin, fibrinogen, albumin, glucose, immunoglobulins, and tryptophan (Altman et al., “Blood and Other Body Fluids,” Washington, D.C.: Federation of American Societies for Experimental Biology (1961), which is hereby incorporated by reference in its entirety). It was hypothesized, that variations in Raman spectra of blood occur due to the changes, related to human's gender.

The averaged, normalized by total area blood spectra of female and male donors have very similar profiles (FIG. 2A). However, small differences can be observed within 930-975, 1210-1230 cm⁻¹ regions, and at 1560, 1580, and 1600 cm⁻¹ Raman peaks. Standard deviation spectra illustrate the heterogeneous nature of blood (FIG. 2B). They are significantly different within the 1200-1300, 1500-1700 cm⁻¹ spectroscopic regions and at 1000 cm⁻¹. The observed level of similarity is consistent with the nearly identical biochemical composition of female and male blood. Furthermore, it is expected that only the few Raman spectra that originated from spots enriched with these characteristic constituents will demonstrate prominent gender-associated spectroscopic features. A preliminary assignment of the major Raman peaks is shown in Table 1.

TABLE 1
Assignment of Raman bands of blood.

| Raman band/cm⁻¹ | Intensity | Assignment^{a} |
|---|---|---|
| 716 | w | γ11 |
| 744 | m | tryptophan |
| 754 | s | ν15 |
| 788 | w | ν6 |
| 900 | w | p: C-C skeletal |
| 937 | m | ν46 |
| 967 | s | lipids, proteins |
| 1002 | s-m | phenylalanine |
| 1030 | w | δ(=CbH2)asym |
| 1054 | w | δ(=CbH2)asym |
| 1122 | s | heme, polysaccharides, |
| | | ν22(porphyrin half |
| | | ring), observed in the |
| | | spectra of single human |
| | | RBC |
| 1173 | w | ν30 |
| 1247 | w | Amide III |
| 1248 | s | guanine, cytosine, |
| | | proteins |
| 1311 | w | ν21 |
| 1342 | m | tryptophan |
| 1368 | | heme |
| 1398 | m | ν20 |
| 1448 | m-w | tryptophan |
| 1542 | | heme |
| 1563 | s | ν19 |
| ~1565 | w | Amide II |
| 1575 | s | DNA bases, proteins |
| 1582 | m | ν37 |
| 1603 | m | ν(Ca = Cb) |
| 1620 | | heme |
| 1638 | m | ν10 |
| 1654 | w | Amide I |

^{a} - Movasaghi et al., “Raman Spectroscopy of Biological Tissues,” Appl. Spectrosc. Rev. 42(5): 493-541 (2007); Alfano et al., Detection of Glucose Levels Using Excitation and Difference Raman Spectroscopy at the IUSL (2008); Janko et al., “Preservation of 5300 Year Old Red Blood Cells in the Iceman,” J. R. Soc. Interface (2012); Aubrey et al., “Raman Spectroscopy of Filamentous Bacteriophage Ff (fd, M13, f1) Incorporating Specifically-Deuterated Alanine and Tryptophan Side Chains. Assignments and Structural Interpretation,” Biophys. J. 60(6): 1337-49 (1991); Grasselli, J., Chemical Applications of Raman Spectroscopy, New York: John Wiley & Sons (1981); Johnson et al., “Ultraviolet Resonance Raman Characterization of Photochemical Transients of Phenol, Tyrosine, and Tryptophan,” J. Am. Chem. Soc. 108: 905-912 (1986); Hu et al., “Tyrosine and Tryptophan Structure Markers in Hemoglobin Ultraviolet Resonance Raman Spectra: Mode Assignments Via Subunit-Specific Isotope Labeling of Recombinant Protein,” Biochemistry 36(50): 15701-12 (1997); Sato et al., “Excitation Wavelength-Dependent Changes in Raman Spectra of Whole Blood and Hemoglobin: Comparison of the Spectra with 514.5-, 720-, and 1064-nm Excitation,” J. Biomed. Opt. 6(3): 366-70 (2001); Premasiri et al., “Surface-Enhanced Raman Scattering of Whole Human Blood, Blood Plasma, and Red Blood Cells: Cellular Processes and Bioanalytical Sensing,” J. Phys. Chem. B, 116(31): 9376-86 (2012), which are hereby incorporated by reference in their entirety.

Main Approach

The present application describes the feasibility of the Raman multidimensional blood signatures from the perspective of donor's sex differentiation. The present application demonstrates that Raman spectra of blood regardless of the gender of donors can be distinguished from other body fluids using earlier developed blood signature (Sikirzhytski, et al., “Multidimensional Raman Spectroscopic Signatures as a Tool for Forensic Identification of Body Fluid Traces: A Review,” Appl. Spectrosc. 65(11):1223-32 (2011), which is hereby incorporated by reference in its entirety).

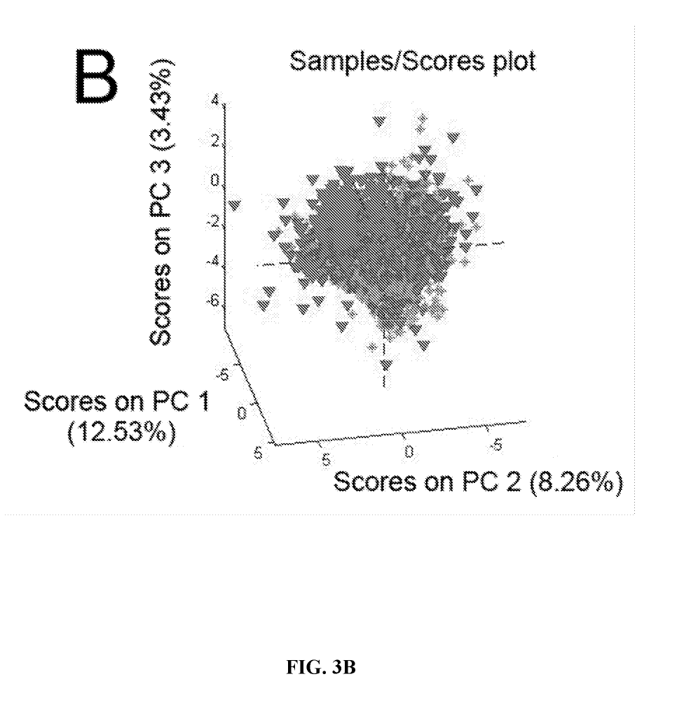

Unsupervised methods of spectroscopic data analysis can be used as a first step of analysis to find out the general relationships between spectra. Their application exposed a high level of similarity between the male and female data sets (FIGS. 3A-B). PCA score plots in FIGS. 3A-B showed highly overlapped female and male data sets with minute space domains dominated only by one single gender. Appearance of such space domains can be tentatively treated as the indication of Raman spectra characteristic of a particular gender. One should keep in mind that patterns observed by unsupervised statistical methods might be a sign of randomness of the spectral data, as well as nonspecific variations between and within donors/samples. Since there was a slight sign of grouping, the following step was establishing the link between the classes of data treatment using a clustering approach. However, extensive validation methods were used to establish the significance of the observations.

Hierarchical clustering methods were used to search for the internal structure of Raman spectroscopic data. This method allows splitting the analyzed data into hierarchical subgroups forming a dendrogram. In particular, spectral clusters unique for male and female donors were under consideration (FIGS. 4A-B). All spectra were organized according to their proximity in the virtual space of PCs, where the closest elements form groups. At this point, it was important to distinguish the basis of clustering of larger groups. As seen in FIGS. 4A-B, KNN clustering using Ward's approach exposed a complex hierarchy of diverse clusters and two of them can be characterized as dominated by “female” (red labels) and “male” (green labels) Raman spectra. All other clusters consisted of Raman spectra of both genders.

Support Vector Machine Discriminant Analysis of Human Blood

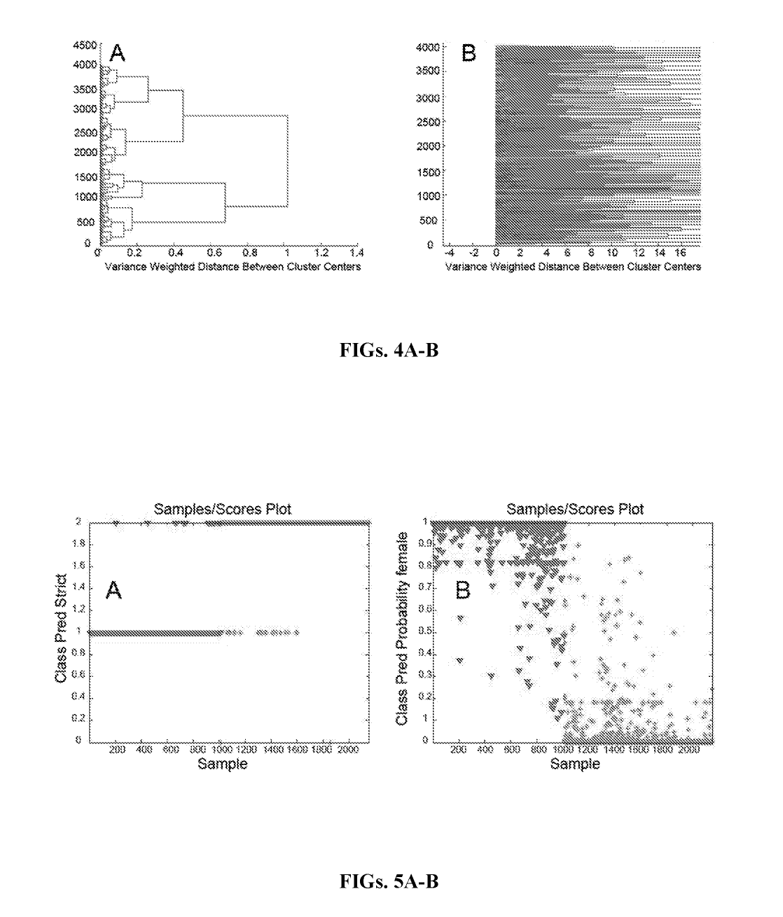

SVMDA classification models built using described characteristic clusters demonstrated high selectivity and sensitivity (˜90%) of gender determinations. Results were cross-validated using sample-wise leave-one-out approach. The best results were obtained using an SVMDA algorithm, which allows for effective separation of overlapping classes (FIGS. 5A-B). Dimensional reduction was performed using PCA. The high selectivity and sensitivity of gender determination verified by cross-validation methods are very encouraging results. The initial selection lead to the significant reduction of datasets.

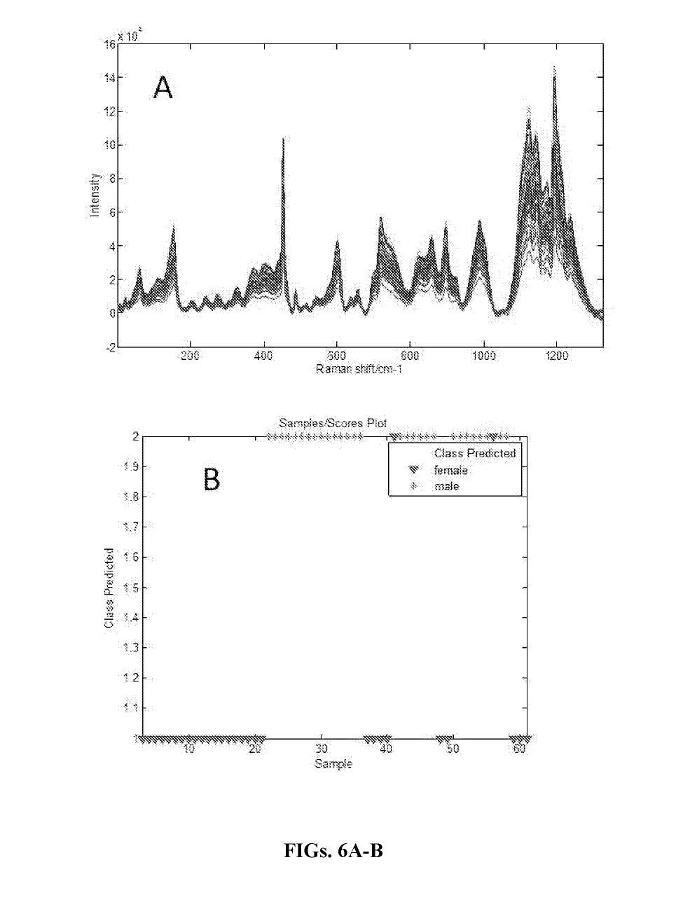

An alternative possibility of data preparation is to calculate averaged spectra and use them for building a classification model. This approach helps to reduce the dimensionality of data and overcome difficulties, originating from the poor quality of some spectra. It was hypothesized that misclassification in different gender classes in some cases can be caused by a relatively low signal-to-noise ratio. The presence of noise influences sensitivity of the method, making spectral features indistinguishable for male and female groups. To overcome this problem, the averaged spectra for each donor were calculated and subjected to SVMDA (FIGS. 6A-B). The averaged spectra were normalized by total area and mean centered prior to discrimination analysis. Ten cross-validation splits were applied to separate data into test and validation subsets. No smoothing procedure was used, since the overall quality of spectra was sufficient. The sensitivity and specificity of the new model were 77% and 93% respectively.

### Conclusions

Raman microspectroscopy was used for the identification of human gender based on dried blood traces. Blood samples from a total of 60 human donors were subjected to automatic mapping followed by chemometrical analysis. Male and female datasets were formed using MATLAB 7.11 after preprocessing (baseline correction, noise reduction and normalization by total area). Spectroscopic patterns from those two groups were found to be the same, despite the high level of blood heterogeneity. Both human genders were described by characteristic Raman spectra based on unsupervised cluster analysis. The most successful results were achieved using the SVM algorithm followed by cross-validation using the sample-wise leave-one-out approach using Raman spectra averaged by donors. Further development of this classification method is ongoing.

### Example 7—Materials and Methods for Example 8

Sample Preparation and Raman Microspectroscopy

Twenty eight human semen samples were purchased from Bioreclamation LLC (Westbury, N.Y.). Donors self-reported their race as Caucasian (n=10), Black (n=8), or Hispanic (n=10). Each group had an age range from mid-twenties to mid-fifties to ensure donor diversity. Samples were kept frozen until preparation for analysis, when they were thawed to room temperature and vortexed for 30 seconds to ensure a homogeneous distribution of the different phases of the sample. A 10 μL aliquot was deposited on an aluminum foil covered microscope slide, which has minimal Raman and fluorescence signal contribution. Samples were air dried overnight prior to analysis.

A Renishaw inVia confocal Raman microspectrometer equipped with a Renishaw PRIOR automatic stage was used for data collection. The excitation source was a 785-nm laser operating at about 50 mW. Calibration was performed with a silicon standard. Spectra were collected with a 50× long range/working distance range objective in the range of 300-1800 cm⁻¹, with a 10 second exposure time and 7 accumulations. Each sample was automatically mapped to collect 64 spectra across an area of approximately 2.0 mm².

Data Treatment

Statistical software MATLAB version R2012a (Mathworks, Inc., Natick Mass.) was used with the PLS Toolbox 7.0.3 (Eigenvector Research, Inc., Wenatchee, Wash.) for data pretreatment and analysis. Spectra that exhibited significant noise or cosmic ray interference were removed from the dataset, resulting in a total of 1,537 spectra. Each sample's dataset was baseline corrected with an adaptive iteratively reweighted penalized least-squares (air-PLS) baseline correction algorithm (Zhang et al., “Baseline Correction Using Adaptive Iteratively Reweighted Penalized Least Squares,” Analyst 135(5):1138-1146 (2010), which is hereby incorporated by reference in its entirety). Spectra were averaged to create one mean spectrum per donor for the development of the model based on donors, instead of individual spectra. The donor's class (Black, Caucasian, or Hispanic) was assigned to all spectra. Two datasets were created from the existing data, one collective dataset with all spectra (n=1,537), and one with all mean spectra (n=28). All spectra were smoothed with a Savitzky-Golay filter, normalized by total area, and mean centered prior to analysis. Principal component analysis (PCA) with leave-one-out cross-validation was applied to the preprocessed collective dataset for dimensionality reduction of the data and to calculate the number of principal components (PCs) that could fully describe the obtained data, which was found to be five. Several comprehensive chemometrical approaches were investigated, including Significant Factor Analysis (SFA), k-nearest neighbor (KNN) hierarchical clustering, Partial Least Squares Discriminant Analysis (PLS-DA), and Support Vector Machine Discriminant Analysis (SVMDA).

### Example 8—Results and Discussion of Example 7

The main objective of this example was to use Raman spectroscopy of dry semen traces to identify a donor's race. Three different classification schemes were explored. First, a chemometric model was built to classify donors into one of the three races (Caucasian, Black, or Hispanic) in one step, based solely on their mean spectrum. Next, a two-step scheme was constructed using the collective data set. The first step classified the spectra into one of the three races studied using a chemometric model, just as the previous model had with the mean spectra. The overall donor classification was then determined using the classification results observed for each individual donor. Finally, a three-step scheme was created. Using the collective dataset, this scheme employed two models to classify the spectra. The first model separated the spectra from Caucasian and Hispanic from those of Black donors. The second model then differentiated Caucasian and Hispanic spectra. In the third and final step, the spectral classification results were used to classify individual donors.

Spectra Acquisition and Analysis

Previously, a spectroscopic signature was reported that can be used to identify semen, and differentiate it from other body fluids (Virkler et al., “Raman Spectroscopic Signature of Semen and its Potential Application to Forensic Body Fluid Identification,” Forensic Sci. Int. 193(1-3):56-62 (2009), which is hereby incorporated by reference in its entirety). The Raman spectrum of dry semen can be characterized by the peaks typical for tyrosine (641, 798, 829, 848, 983, 1179, 1200, 1213, 1265, 1327, and 1616 cm⁻¹), choline (715 cm⁻¹), albumin (759, 1003, 1336, and 1448 cm⁻¹), other proteins (1668 and 1240 cm⁻¹), and spermine phosphate hexahydrate (888, 958, 1011, 1055, 1065, 1125, 1317, 1461, and 1494 cm⁻¹) (Sikirzhytski et al., “Multidimensional Raman Spectroscopic Signatures as a Tool for Forensic Identification of Body Fluid Traces: A Review,”Applied Spectroscopy 65(11):1223-32 (2011), which is hereby incorporated by reference in its entirety).

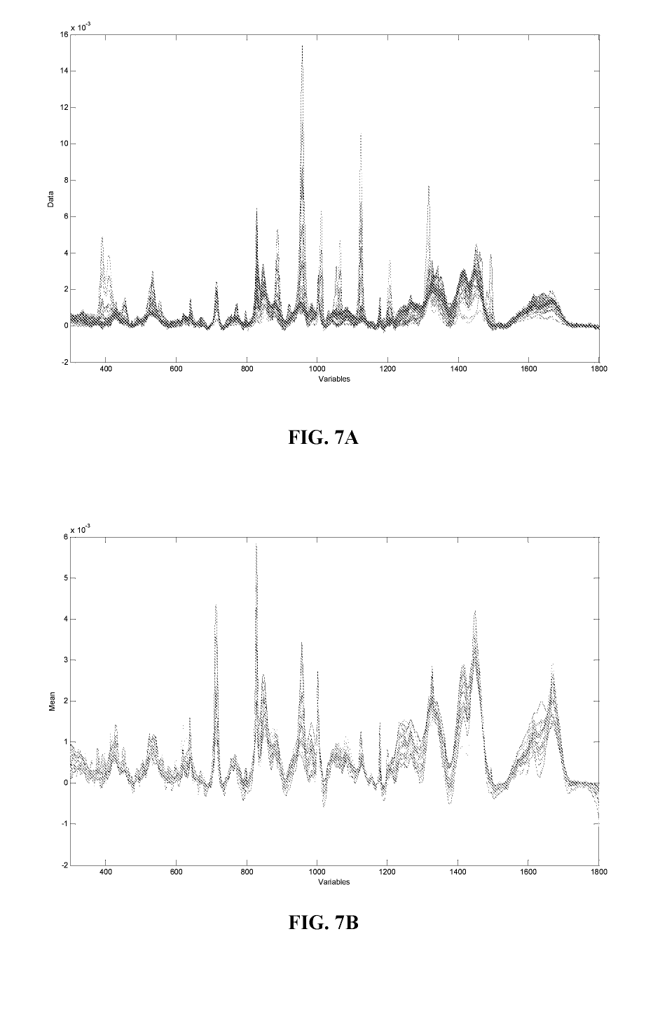

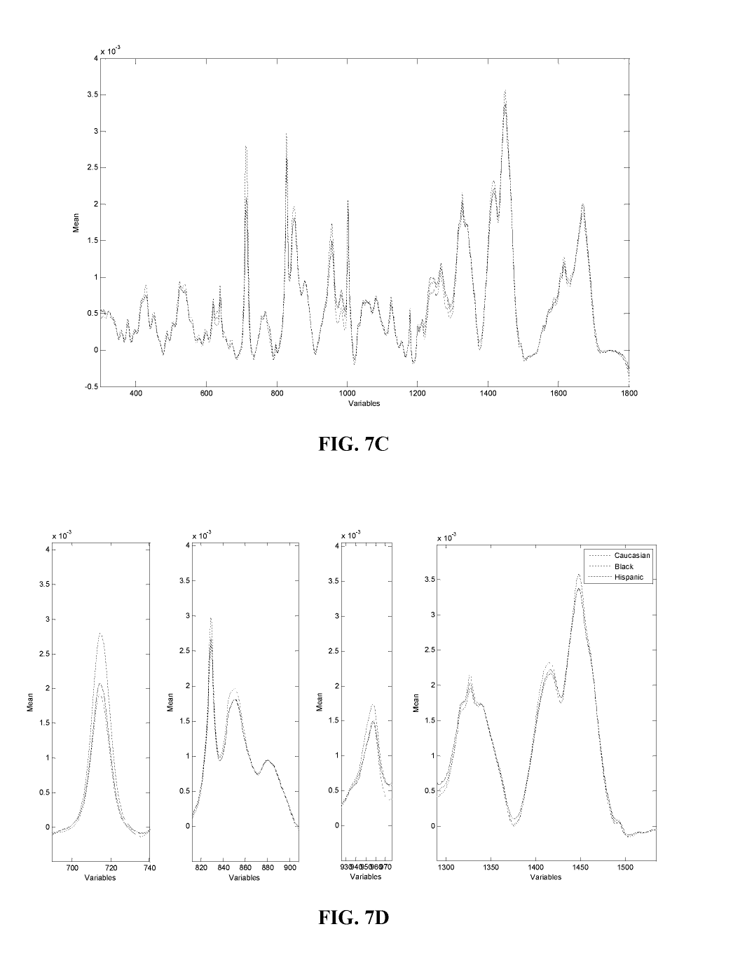

The spectra showed significant variation between donors and within the same sample, illustrating semen's heterogeneous nature (FIG. 7A). Despite the high level of heterogeneity, when each donor's spectra were averaged the mean spectra showed consistency in major peak positions and shapes (FIG. 7B). Subtle differences were observed when the mean spectra of the three races, Black, Caucasian, and Hispanic, were compared (FIGS. 7C-D). For example, the average spectrum for all Caucasian donors has the highest intensity at 715, 957, and 1448 cm⁻¹. Conversely, Black samples, on average, have the highest intensity at 829, 851, 1327, and 1415 cm⁻¹.

One-Step Classification Scheme

Several different decomposition, regression, and classification models were investigated. An SVMDA model proved to be the best at differentiating the races, based on true positive and true negative rates. The SVMDA model parameters were optimized to enhance classification performance. The first SVMDA model was built using the 28 mean spectra, as a way to classify at the individual level as opposed to the spectral level. As a result, the model generated would classify donors in a single step. Unfortunately, this approach did not yield successful results; 18 of the 28 donors were misclassified (FIG. 8 and Table 2).

TABLE 2
The cross-validated true positive and true negative and error rates of
the SVMDA model built using the mean data set.

| | Caucasian | Black | Hispanic |
|---|---|---|---|
| True Positive (CV) | 0.500 | 0.250 | 0.300 |
| True Negative (CV) | 0.778 | 0.800 | 0.444 |

| | |
|---|---|
| RMSECV | 0.142857 |

Two-Step Classification Model

Based on the results from the direct application of the classification algorithm on the mean spectra, it was hypothesized that the collective dataset may yield more accurate predictions. When the donor's spectra are averaged, it can mask subtle, but key, spectral features that are characteristic of certain races. In a study from Belgium, researchers attempted to differentiate human, canine, and feline blood using an average spectrum and no statistical analysis (De Wael et al., “In Search of Blood—Detection of Minute Particles using Spectroscopic Methods,” Forensic Sci. Int. 180(1):37-42 (2008), which is hereby incorporated by reference in its entirety). In another study, these exact groups were differentiated using Raman mapping and chemometric models (Virkler et al., “Blood Species Identification for Forensic Purposes using Raman Spectroscopy Combined with Advanced Statistical Analysis,” Anal. Chem. 81(18):7773-7777 (2009), which is hereby incorporated by reference in its entirety).

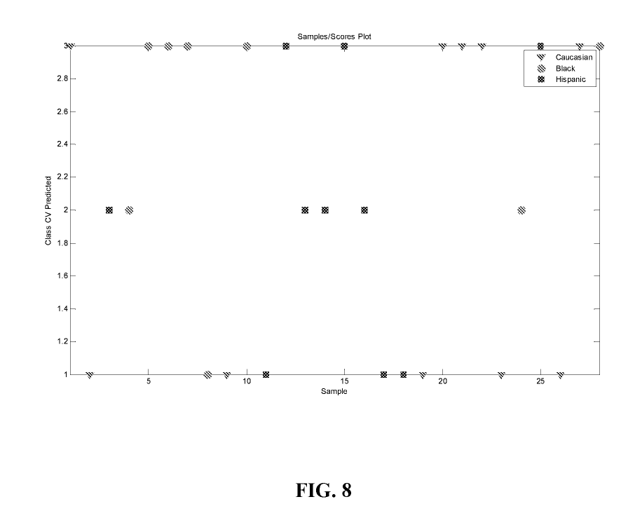

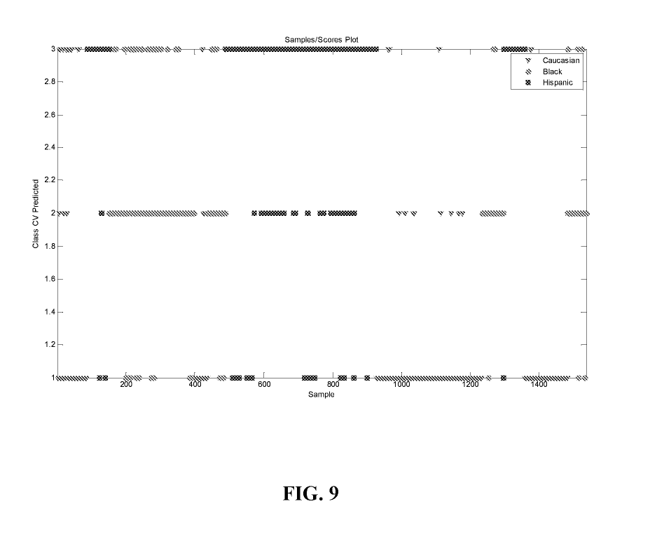

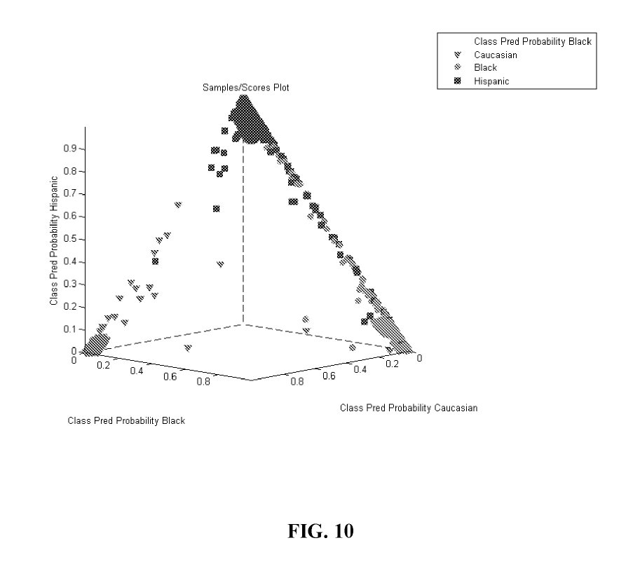

The SVMDA model in the two-step system was built using the collective data set. The results from the model and its classification sensitivity and specificity are shown in FIG. 9 and Table 3, respectively. FIG. 10 shows a score plot for the class prediction probability obtained for individual spectra based on the SVMDA model.

TABLE 3
The Cross-Validated True Positive and True Negative and Error Rates
of the SVMDA Model Built on the Collective Dataset.

| | Caucasian | Black | Hispanic |
|---|---|---|---|
| True Positive (CV) | 0.939 | 0.866 | 0.892 |
| True Negative (CV) | 0.966 | 0.950 | 0.936 |

| | |
|---|---|
| RMSECV | 0.0904359 |

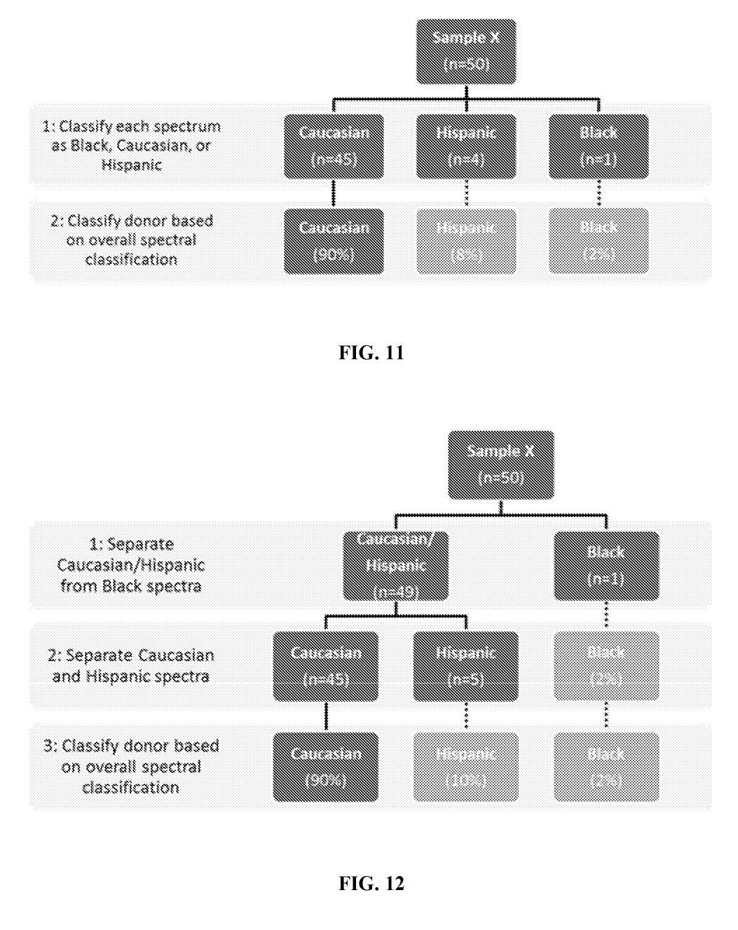

While the model's classification performance was improved by using the collective dataset, a complication was presented. In the mean dataset, each donor was represented by a single spectrum, so the SVMDA model classified each donor into one race. The model was built using the collective dataset, where each donor was represented by several spectra, could classify some number of a single donor's spectra into more than one race. Therefore, this approach can lead to ambiguous results. To resolve this problem, a classification scheme was developed to use the results from the SVMDA model to classify individuals on the donor level (FIG. 11).

Using this classification scheme, the donor classification results were significantly better than the results from the first SVMDA model, which was built using the mean spectra. When every donor is studied individually, on average 90% of each donor's spectra were classified correctly (Table 4). Table 4 shows the breakdown of each donor's spectral classification, including the number and percentage classified correctly. A threshold was set at 51%, such that if 51% of a donor's spectra were attributed to a specific race, the donor was classified as a member of that race. Using this threshold, 100% of donors were classified into the correct race (Table 5). This is a notable improvement from the first SVMDA model built using the mean spectra, which only classified 10 (35.7%) donors into the correct race.

TABLE 4
Results From Two-Step Classification System.

| | Actual | | Predicted | | | | |
|---|---|---|---|---|---|---|---|
| | | Spectra | Caucasian | Black | Hispanic | Correct | |
| Donor | Race | n | n | n | n | n | % |
| 1 | Caucasian | 42 | 27 | 3 | 12 | 27 | 64% |
| 2 | Caucasian | 44 | 42 | 0 | 2 | 42 | 95% |
| 3 | Hispanic | 64 | 2 | 2 | 60 | 60 | 94% |
| 4 | Black | 48 | 0 | 44 | 4 | 44 | 92% |
| 5 | Black | 45 | 6 | 27 | 12 | 27 | 60% |
| 6 | Black | 48 | 2 | 38 | 8 | 38 | 79% |
| 7 | Black | 60 | 0 | 56 | 4 | 56 | 93% |
| 8 | Black | 46 | 2 | 43 | 1 | 43 | 93% |
| 9 | Caucasian | 39 | 36 | 2 | 1 | 36 | 92% |
| 10 | Black | 52 | 2 | 43 | 7 | 43 | 83% |
| 11 | Hispanic | 49 | 4 | 0 | 45 | 45 | 92% |
| 12 | Hispanic | 58 | 2 | 3 | 53 | 53 | 91% |
| 13 | Hispanic | 64 | 0 | 15 | 49 | 49 | 77% |
| 14 | Hispanic | 50 | 0 | 3 | 47 | 47 | 94% |
| 15 | Hispanic | 57 | 5 | 3 | 49 | 49 | 86% |
| 16 | Hispanic | 38 | 0 | 4 | 34 | 34 | 89% |
| 17 | Hispanic | 60 | 3 | 13 | 44 | 44 | 73% |
| 18 | Hispanic | 63 | 1 | 0 | 62 | 62 | 98% |
| 19 | Caucasian | 64 | 61 | 0 | 3 | 61 | 95% |
| 20 | Caucasian | 53 | 49 | 4 | 0 | 49 | 92% |
| 21 | Caucasian | 63 | 63 | 0 | 0 | 63 | 100% |
| 22 | Caucasian | 63 | 59 | 3 | 1 | 59 | 94% |
| 23 | Caucasian | 64 | 63 | 1 | 0 | 63 | 98% |
| 24 | Black | 62 | 1 | 59 | 2 | 59 | 95% |
| 25 | Hispanic | 62 | 1 | 0 | 61 | 61 | 98% |
| 26 | Caucasian | 62 | 60 | 0 | 2 | 60 | 97% |
| 27 | Caucasian | 61 | 61 | 0 | 0 | 61 | 100% |
| 28 | Black | 56 | 2 | 51 | 3 | 51 | 91% |

TABLE 5
The Cross-Validated True Positive and True Negative and Error Rates
of the SVMDA Models in the Three-Step Classification System.

| 1^{st} SVMDA Model | Caucasian/Hispanic | Black |
|---|---|---|
| True Positive (CV) | 0.963 | 0.863 |
| True Negative (CV) | 0.863 | 0.963 |

| | |
|---|---|
| RMSECV | 0.057905 |

| 2^{nd} SVMDA Model | Caucasian | Hispanic |
|---|---|---|
| True Positive (CV) | 0.939 | 0.965 |
| True Negative (CV) | 0.965 | 0.939 |

| | |
|---|---|
| RMSECV | 0.0410714 |

Table 4 shows that while the classification results given by the model were not perfect, every donor clearly fell into one race. In each case, a majority of spectra were correctly classified into one race, with only a few being misclassified. On average, 90% of each donor's spectra were classified correctly. This shows that most of the samples were not being classified by a simple majority, but rather by an overwhelming proportion.

Three-Step Classification System

While all donors were separated with 100% accuracy using the two-step classification scheme, the SVMDA model used did yield perfect results. Upon closer examination of the misclassified data, it was observed that a majority were from Caucasian or Hispanic donors. In an attempt to improve the average number of spectra classified correctly a third approach was investigated. A three-step classification system was designed, the first two steps consisted of SVMDA models to classify the spectra and the third step classified the donors (FIG. 12). The first SVMDA model separated Caucasian and Hispanic spectra from Black spectra. The second model differentiated Caucasian and Hispanic spectra. From these results, the donors' race was determined.

The results from the models are reported in Table 5. The true positive and true negative rates are similar to those reported for the two-step system, but the error has decreased considerably. The classification results from the first and second SVMDA models are reported in Table 6. Using the same 51% threshold applied in the second classification system, the third system also classifies all 28 donors correctly.

TABLE 6
Classification Results From the First and Second SVMDA
Models in the Three-Step Classification System.

| | | | 1: Black vs. Caucasian/Hispanic | | | | 2: Caucasian vs. Hispanic | | | |
|---|---|---|---|---|---|---|---|---|---|---|
| | | | Black | Cauc/Hisp | Classified | | Caucasian | Hispanic | Classified | |
| Donor | Race | Spectra | n | n | n | % | n | n | n | % |
| 1 | Caucasian | 42 | 4 | 38 | 38 | 90% | 27 | 15 | 27 | 64% |
| 2 | Caucasian | 44 | 1 | 43 | 43 | 98% | 41 | 3 | 41 | 93% |
| 3 | Hispanic | 64 | 0 | 64 | 64 | 100% | 2 | 62 | 62 | 97% |
| 4 | Black | 48 | 45 | 3 | 45 | 94% | — | — | — | — |
| 5 | Black | 45 | 35 | 10 | 35 | 78% | — | — | — | — |
| 6 | Black | 48 | 57 | 14 | 34 | 71% | — | — | — | — |
| 7 | Black | 60 | 43 | 3 | 57 | 95% | — | — | — | — |
| 8 | Black | 46 | 1 | 3 | 43 | 93% | — | — | — | — |
| 9 | Caucasian | 39 | 40 | 38 | 38 | 97% | 35 | 4 | 35 | 90% |
| 10 | Black | 52 | 7 | 12 | 40 | 77% | — | — | — | — |
| 11 | Hispanic | 49 | 11 | 49 | 49 | 100% | 4 | 45 | 45 | 92% |
| 12 | Hispanic | 58 | 6 | 51 | 51 | 88% | 3 | 55 | 55 | 95% |
| 13 | Hispanic | 64 | 1 | 53 | 53 | 83% | 3 | 61 | 61 | 95% |
| 14 | Hispanic | 50 | 0 | 44 | 44 | 88% | 1 | 49 | 49 | 98% |
| 15 | Hispanic | 57 | 2 | 56 | 56 | 98% | 4 | 53 | 53 | 93% |
| 16 | Hispanic | 38 | 0 | 38 | 38 | 100% | 0 | 38 | 38 | 100% |
| 17 | Hispanic | 60 | 2 | 58 | 58 | 97% | 2 | 58 | 58 | 97% |
| 18 | Hispanic | 63 | 2 | 63 | 63 | 100% | 1 | 62 | 62 | 98% |
| 19 | Caucasian | 64 | 1 | 62 | 62 | 97% | 59 | 5 | 59 | 92% |
| 20 | Caucasian | 53 | 2 | 51 | 51 | 96% | 51 | 2 | 51 | 96% |
| 21 | Caucasian | 63 | 1 | 62 | 62 | 98% | 63 | 0 | 63 | 100% |
| 22 | Caucasian | 63 | 61 | 61 | 61 | 97% | 61 | 2 | 61 | 97% |
| 23 | Caucasian | 64 | 0 | 63 | 63 | 98% | 61 | 3 | 61 | 95% |
| 24 | Black | 62 | 1 | 1 | 61 | 98% | — | — | — | — |
| 25 | Hispanic | 62 | 0 | 62 | 62 | 100% | 0 | 62 | 62 | 100% |
| 26 | Caucasian | 62 | 45 | 61 | 61 | 98% | 62 | 0 | 62 | 100% |
| 27 | Caucasian | 61 | 0 | 61 | 61 | 100% | 61 | 0 | 61 | 100% |
| 28 | Black | 56 | 0 | 11 | 45 | 80% | — | — | — | — |

In the first SVMDA model, 21 (75%) of the 28 donors have at least 90% of their spectra classified correctly. In the second step, 19 (95%) of the 20 donors have at least 90% of their spectra classified correctly. The overall trend is not just a simple majority being classified correctly, but that the models are classifying a vast majority of each donor's spectra correctly. On average, only 5% of each donor's spectra were misclassified in the first step, and only 5% in the second step.

For the three-step classification system, donor #1 demonstrated the lowest rate of classification in the second SVMDA model. While 90% of this donor's spectra were classified correctly as Caucasian/Hispanic in the first step, only 64% were classified correctly as Caucasian in the second step. Bioreclamation LLC was contacted to request additional information about this particular donor. More detailed records showed that the donor was actually biracial, of both Caucasian and Hispanic descent. Although this information provides a possible explanation as to why this particular donor had poor classification rates, it also introduces a new limitation. Semen from biracial or mixed-race men may prove to be more difficult to classify. However further data collection, from additional biracial donors, could be used to investigate this unique class more thoroughly. Eventually, new classes could be added to the model to differentiate these samples as well.

### Conclusions

Near-Infrared (NIR) Raman microspectroscopy was used to analyze human semen samples.

A new two-step classification system using advanced statistical analysis was developed to determine a donor's race based on the Raman spectroscopic profile of their semen. An SVMDA model was used to classify each spectrum as belonging to one of the three races studied, Caucasian, Black, or Hispanic. The sensitivity and specificity scores for the model were reported as 93.9/86.6/89.2 and 96.6/95.0/93.6, respectively.

A new three-step classification system using advanced statistical analysis was developed to determine a donor's race based on the Raman spectroscopic profile of their semen. Two SVMDA models were used in sequence to classify each spectrum as belonging to one of three races. The sensitivity/specificity of the first and second model was 96.3/86.3% and 93.9/96.5%, respectively.

The overall classification pattern of each donor's spectra was used to classify the individual's race. This final step resulted in 100% sensitivity and specificity. The results obtained during the SVMDA classification were examined using extensive cross-validation with spectroscopic data acquired from additional donors. The small amount of sample needed, minimal sample preparation, automated scanning, and nondestructive nature of this method give it the potential to be very useful in forensic investigations. The present model can be further improved by including more racial groups, analyzing more samples from biracial donors, and acquiring samples for external validation. Nonetheless, the method demonstrates the ability of Raman spectroscopy and advanced statistical analysis to determine an individual's race from their semen. The present method can be extended by including more racial groups as well as differentiation of donors by their age.

### Example 9—Materials and Methods for Example 10

Blood Samples

The experiment was performed on human blood collected from 30 donors in total which was acquired from Bioreclamation, Inc. Samples were divided into gender (15 per subset) and race (10 per each including CA, AA and HI) classes. Age diversity was maintained in subject selection. From the total sample population, 26 were used to create a training dataset. The remaining four samples were used as blind samples to externally validate the models built. Each blood sample was defrosted and vortexed to obtain its homogeneous content before deposition. Samples were prepared by depositing 30 μL of fluid on microscope slide for overnight drying.

Instrumentation and Spectra Collection

Spectra were recorded using a PerkinElmer Spectrum 100 FT-IR Spectrometer connected with Spectrum software version 6.0.2.0025 (PerkinElmer, Inc.). A diamond/ZnSe plate was used as an ATR attachment which was cleaned with water and acetone before each sample, and a 10% bleach solution after each analysis. Consistently, a background check was run prior to collecting spectra. Ten spectra were recorded from each sample in a spectral range of 600-4000 cm⁻¹. Each spectrum was the result of ten co-added scans. The spectral resolution was set to 4 cm⁻¹.

Data Treatment

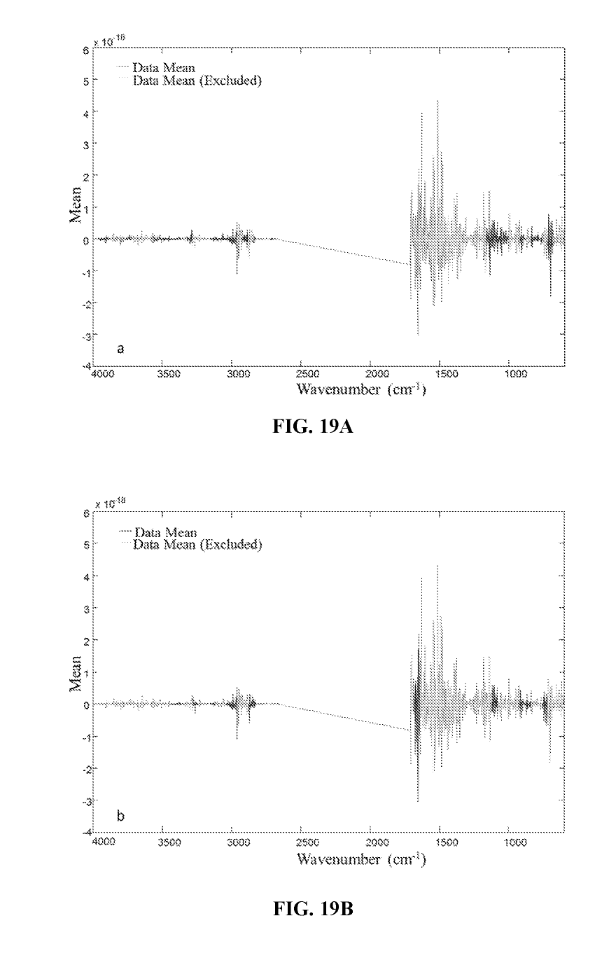

Dataset preparation and statistical analysis was performed using MATLAB (Mathworks, Inc. version R2013b) with PLS Toolbox (Eigenvector Research, Inc.) (Wise et al., PLS_Toolbox 3.5 for Use with MATLAB Wenatchee, Wash.: Eigenvector Research, Inc. (2005), which is hereby incorporated by reference in its entirety). Previous studies on species' differentiation based on infrared blood spectra demonstrated enhanced contribution from the ATR crystal in the spectral range of 1711-2669 cm⁻¹ (FIG. 18). Accordingly, this region was excluded from the spectra. All the collected spectra with the excluded background region were preprocessed by applying transmission to absorbance transformation (log(1/T)), 2nd order derivative with a second polynomial for smoothing and baseline correction, normalization by total area and mean centering (Rinnan et al., “Review of the Most Common Pre-Processing Techniques for Near-Infrared Spectra,” TrAC Trends in Anal. Chem. 28(10):1201-1222 (2009), which is hereby incorporated by reference in its entirety). After these preprocessing steps, GA was employed to select the most significant variables or set of variables for classifying the applied classes (FIGS. 19A-B). GA was run in two different ways, to determine the spectral ranges most valuable for gender identification and for distinction between races. PCA models were created for both datasets (gender and race). This allowed for detecting outliers; spectra with the most abnormal Hotelling T2 and Q residuals (Rodriguez et al., “Raman Spectroscopy and Chemometrics for Identification and Strain Discrimination of the Wine Spoilage Yeasts Saccharomyces Cerevisiae, Zygosaccharomyces Bailii, and Brettanomyces Bruxellensis,” Appl. Environ. Microbiol. 79(20):6264-6270 (2013), Xiao et al., “Drift Compensation of Gas Sensor Array by Matrix Transform and Genetic Algorithm Based on Database,” J. Computational Information Systems, 9(9):3469-3476 (2013), which are hereby incorporated by reference in their entirety). A supervised statistical method, PLS-DA, was employed to discriminate males and females, as well as races (Varmuza et al., Introduction to Multivariate Statistical Analysis in Chemometrics. CRC Press (2008), which is hereby incorporated by reference in its entirety). The ROC analysis was performed with the open source package pROC (Robin et al., “pROC: An Open-Source Package for R and S+ to Analyze and Compare ROC Curves.” BMC Bioinformatics 12(1):77 (2011), which is hereby incorporated by reference in its entirety). The ROC analysis was utilized to assess the discriminatory power of the PLS classifier and select the best threshold. To indicate how well the model ranks subjects according to the probability assigned to the correct class, the AUC analysis was performed.

Validation Tests

Because of the small sample population size, a large enough test (blind) dataset was not created after using 26 of the 30 donors in the calibration dataset. In order to achieve the best verification of this model, the PLS-DA model was validated in two different ways. Firstly, to rule out the effect of the test dataset size, the training dataset consisting of 26 donors was externally cross-validated where the spectra from one donor were removed from the training dataset and the PLS-DA model was refit to remaining training data, and used to predict the corresponding test set, which had been removed. This was repeated until all subjects were removed and predicted. No subjects that were used to test predictions were used during the model development, so a reliable error rate of CV was ensured (Anderssen et al., “Reducing Over-Optimism in Variable Selection by Cross-Model Validation,” Chemometrics and Intelligent Laboratory Systems 84(1-2):69-74 (2006), which is hereby incorporated by reference in its entirety). CV results are reported as the performance over all test sets. This provided an estimate of model performance, and it confirmed classification of predictions performed for this particular training dataset. However, it required refitting the model for each individual subject. Therefore, generalizability and predictive potential given by the external CV was additionally assessed by validation of the primary PLS-DA models with all 26 donors by predicting four blind samples that had been separated from training dataset from the beginning of the statistical analysis. The Y values for all spectra were predicted by building PLS-DA models using the same number of optimal latent variables as was determined by CV. For each class prediction and its corresponding PLS-DA classifier the threshold was determined. The trained threshold of Y predictions identified during the external CV was used to classify gender or race of all test samples. During the testing, the features extracted from spectra were compared against the trained threshold to assess the gender and race assignment. The test samples included a diversity of gender and race (1 CA male, 1 AA male, 1 CA female, 1 HI female). This step was used to examine the prediction performance of the method and models, as well as to confirm the models' integrity when analyzing external, unknown bloodstains.

### Example 10—Results and Discussion of Example 9

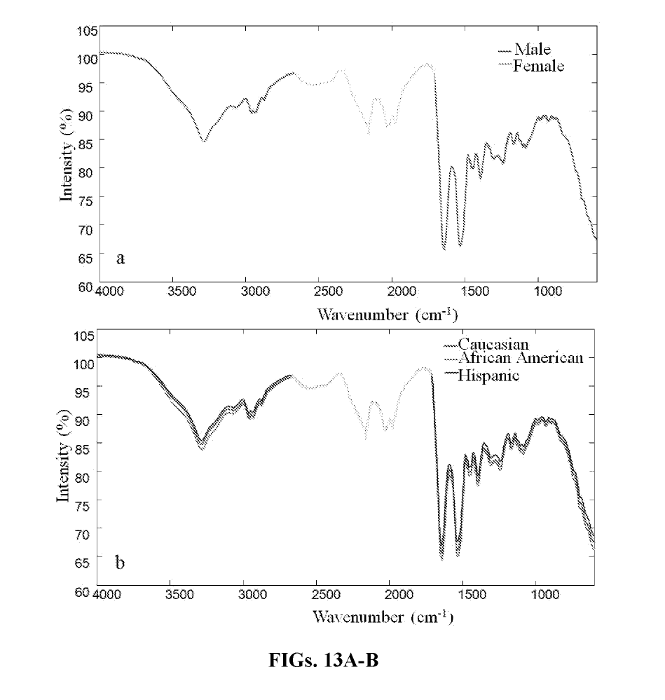

Discrimination of blood donors is possible based on differences in concentration of blood components between groups (Virkler et al., “Raman Spectroscopic Signature of Blood and its Potential Application to Forensic Body Fluid Identification,” Anal. Bioanal. Chem. 396(1):525-534 (2010), which is hereby incorporated by reference in its entirety). The main approach of this example was to develop ATR-FTIR spectroscopy as an analytical technique capable of detecting these changes. Although all blood infrared spectra looked very similar, as can be seen in FIGS. 13A-B and previous studies (De Wael et al., “In Search of Blood—Detection of Minute Particles Using Spectroscopic Methods,” Forensic Sci. Int. 180(1):37-42 (2008), which is hereby incorporated by reference in its entirety), the identification process was enhanced by chemometric analysis and showed promise. This approach showed its potential utility in real crime scene investigation for collecting additional information about a bloodstain to narrow the area of examination without causing any harm to the sample (Robotham et al., “FT-IR Microspectroscopy in Forensic and Crime Lab Analysis,” Thermo Fisher Scientific. Madison Wis. USA (2002), which is hereby incorporated by reference in its entirety). The entire analysis can be performed and results can be obtained at a crime scene due to the accessibility of portable FTIR instruments (Mukhopadhyay, R., “Product Review: Portable FTIR Spectrometers Get Moving,” Anal. Chem. 76(19):369 A-372 A (2004), which is hereby incorporated by reference in its entirety).

Spectral Analysis and Training Dataset

Blood infrared spectra (FIGS. 13A-B) were found to be very similar; the number of peaks was equivalent and they were located in the same position. The spectral region of 1711-2669 cm⁻¹ was caused by interference from the diamond ATR crystal, so it was excluded from all spectra. The major spectral features detected in the IR spectra of biological samples were observed, such as lipids (3100-2800 cm⁻¹), proteins (1800-1400 cm⁻¹), and carbohydrates (1400-900 cm⁻¹) (Kanagathara et al., “FTIR and UV-Visible Spectral Study on Normal Blood Samples,” Int. J. Pharm. Biol. Sci. 1:74-81 (2011); Baker et al., “Using Fourier Transform IR Spectroscopy to Analyze Biological Materials,” Nat. Protocols 9(8):1771-1791 (2014), which are hereby incorporated by reference in their entirety). Due to the inability to visually identify blood spectra, advanced statistical analysis was required. Spectral regions showing the biggest differences between classes for both sets were detected by GA (FIGS. 19A-B). Within selected spectral regions some peaks corresponding to the molecular vibrations can be identified (Table 7).

TABLE 7
Assignment of the Infrared Bands of Human Blood.

| Wavenumber (cm⁻¹) | Assignment^{a} |
|---|---|
| 699 | C—H bending of amide IV |
| 1078 | C—O symmetric stretching of glucose |
| 1160 | C—O stretching |
| 1600-1700 | C═O symmetric stretching of amide I |
| 2870 | symmetric stretching of CH3 |
| 2960 | asymmetric stretching of CH3 |
| 3200-3500 | O—H stretching of water and hydroxyls |

^{a}Kanagathara et al., “FTIR and UV-Visible Spectral Study on Normal Blood Samples,” Int. J. Pharm. Biol. Sci. 1: 74-81 (2011); Elkins, K. M., “Rapid Presumptive “Fingerprinting” of Body Fluids and Materials by ATR FT-IR Spectroscopy,” J. Forensic Sci. 56(6): 1580-1587 (2011), which are hereby incorporated by reference in their entirety.

Collected blood infrared spectra were assigned in two ways for gender and race differentiation. After spectra were preprocessed and analyzed by GA for variable selection, they were used to build a PCA model to detect outliers and exclude them from the classification process. For this purpose, Hotelling T2 and Q residuals analyses were used (Varmuza et al., Introduction to Multivariate Statistical Analysis in Chemometrics. CRC Press (2008), which is hereby incorporated by reference in its entirety). This made it possible to limit the dataset to the spectra which were not influenced by divergence within the dataset. Subsequently, selected spectra were used to construct a new set, which was divided into a training dataset (26 donors) and an external, blind, dataset (4 donors). Different models were created based on the training set for classification processes. PLS-DA was chosen as the most applicable model for use in predictions, which was determined based on the results of internal prediction performance obtained by the models, as well as the lowest error values of created models. The next step of this approach was validation tests.

Validation Tests for Gender

The first step of validation was external CV. The spectra from one subject were removed from the original calibration dataset, a new PLS-DA model was built, and the previously excluded spectra were used to test the new model. Repeating the process in the manner that each subject appears once in validation set, class labels of all subjects were predicted. Based on these predictions for all 26 donors (13 per class of male and female) contained in the training dataset, AUC and number of misclassifications was obtained. Prediction performance of the PLS-DA models is measured by ROC (FIGS. 14A-B) where AUC achieved was 0.81 (95% CI: 0.75-0.86) and 0.91 (95% CI: 0.78-1.00) based on a single spectrum and each subject, respectively. It confirmed classification performance of predictions for the approach and lead us to complete external validation with the blind test samples.

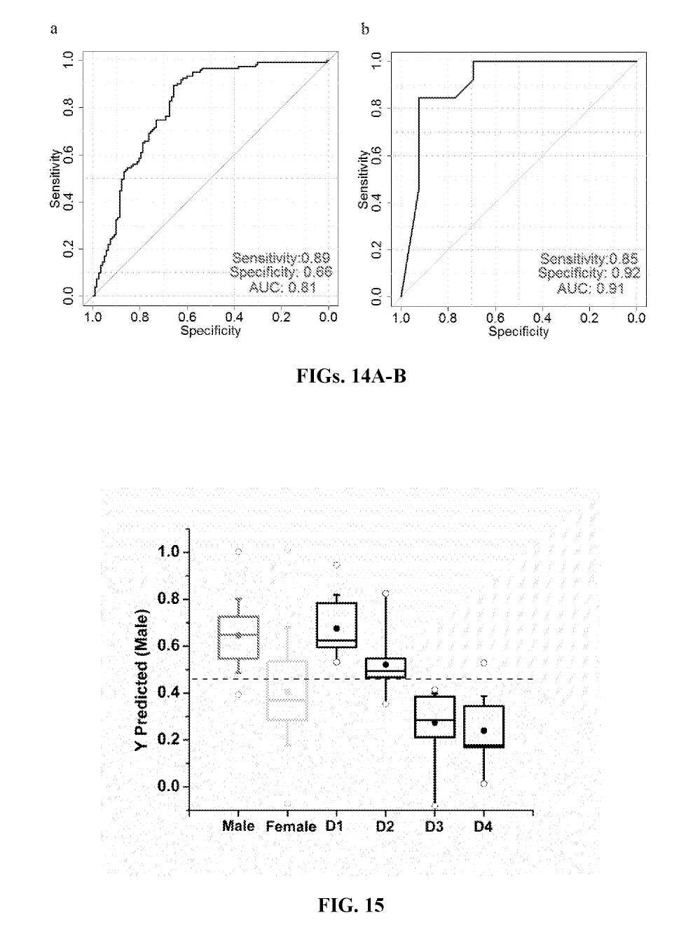

For the next validation step, the model was built on the original 26 samples using an optimal number of components. Next, the class labels of all blind spectra of four donors were predicted using the model and trained threshold. Of the 39 spectra collected from the four blind samples, 36 were classified correctly (FIG. 15). When the classification patterns of each blind sample's spectra are studied at the donor level, it could be seen that all four individuals are classified correctly. Using this approach, the versatility and the high performance of optimized PLS-DA model for determination of gender based on FTIR spectra were demonstrated.

Validation Tests for Race

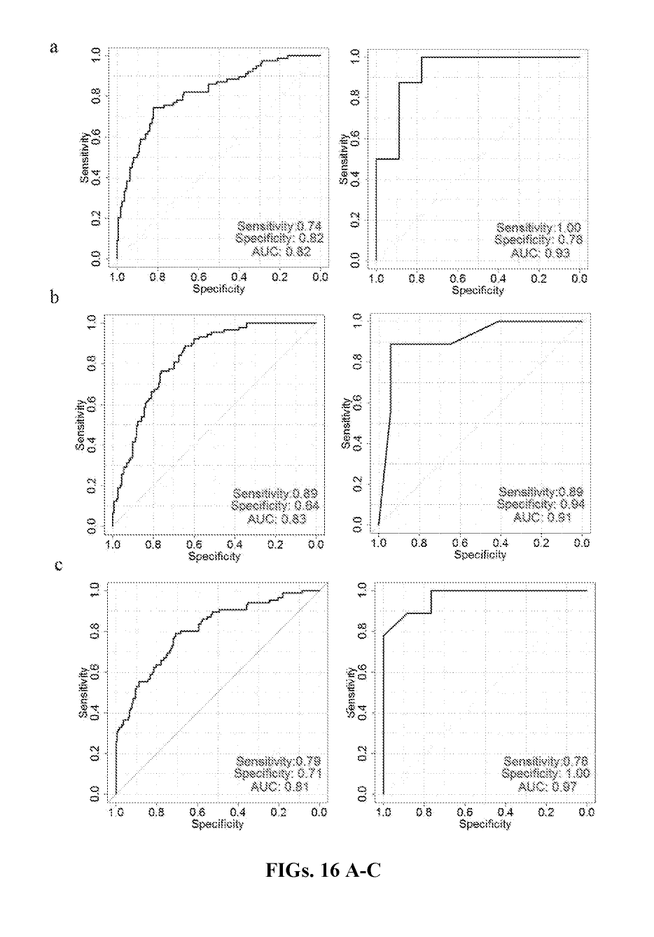

Spectra separated into racial groups (10 donors per class) were treated in the same manner as dataset used for external CV in gender predictions. Based on these predictions of all 26 donors included in training dataset, AUC and number of misclassification were obtained. Prediction performance of the PLS-DA models was assessed by ROC (FIGS. 16A-C). AUC based on a single spectrum achieved values of 0.82 (95% CI: 0.76-0.88), 0.83 (95% CI: 0.78-0.88) and 0.81 (95% CI: 0.75-0.86) of the CA, AA and HI PLS-DA models, respectively. It validated classification performance of predictions for the approach and enabled complete subsequent validation of blind test samples.

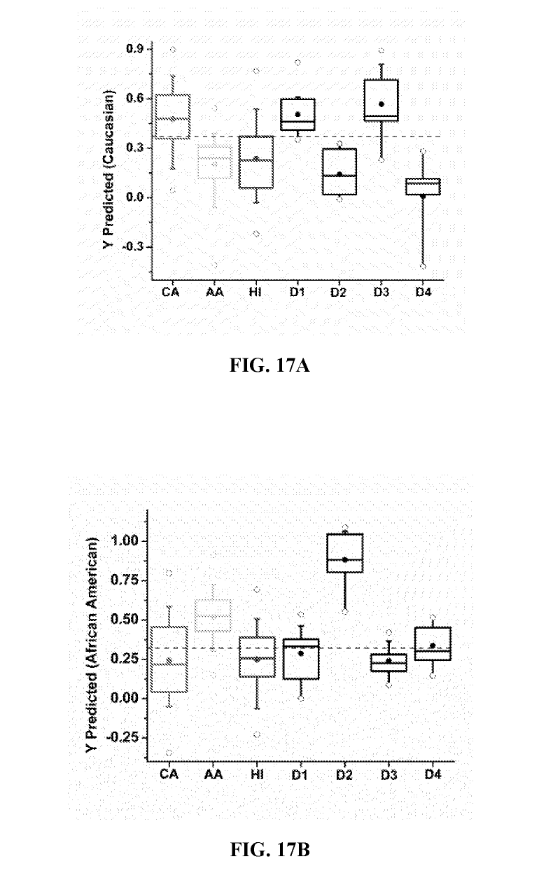

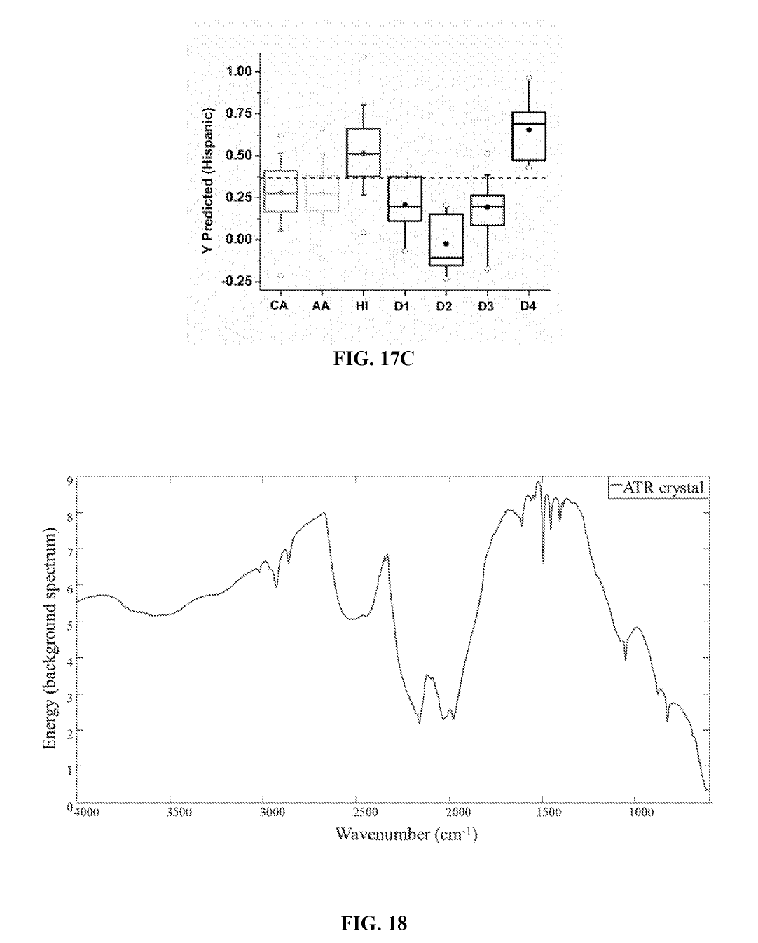

The class labels of all blind spectra of four donors were predicted in the same manner as a gender dataset. Class labels were predicted again with 36 of 39 blind spectra correctly classified and all donors were classified correctly. Using this approach, the versatility and the high performance of optimized PLS-DA models was also demonstrated for determination of race based on FTIR spectra (FIGS. 17A-C).

### Conclusions

FTIR spectroscopy has already been utilized in forensic laboratories for drug analysis. Application of this approach for other forms of evidence would be very valuable, including cost reduction, among others. Its nondestructive nature is one of the most desirable in forensic investigations since examined traces can be still subjected to further analysis. The problem of minuscule sizes of trace evidence found at crime scenes can be resolved by this aspect. The method does not require protein extraction, like most current forensic methods for bloodstain analysis, in order to gain information about the donor. In this study, infrared spectra were collected from 30 donors in total. PLS-DA classification models were successfully utilized for discrimination between genders, which resulted with 91% probability of donors' correct classification, and races, which resulted with 94% on average probability of donors' correct classification based on external CV. The main classification models were also validated with four external blind samples giving 100% accuracy for each donor's classification. The combination of FTIR spectroscopy with chemometrics showed a great ability for human gender and race discrimination from dry blood traces in forensic analysis. FTIR portable instruments facilitate investigation and allow for obtaining results at a crime scene.

### Example 11—Determine Race and Gender Based on Saliva

### Summary

In order to investigate the ability to differentiate gender and race using Raman spectra collected from saliva samples, a proof of concept study was designed and implemented. Saliva samples from 60 donors were analyzed by Raman spectroscopy and chemometrics. Two SVM-DA models were built using preprocessed spectra. The models classified the spectra according to race (Caucasian, Black, or Asian) and gender (male or female). The average accuracy of the race differentiation model was 65.4%, and the average accuracy of the gender differentiation model was 82.8%.

Experimental Work

Saliva samples were purchased from Biological Specialty Corp. and Lee BioSolutions. The sample population included saliva from 60 donors, with an equal number of male and female subjects. The 60 donors represented four racial groups, Caucasian (n=20), Black (n=20), and Asian (n=20). All samples were prepared by depositing 104 onto a microscope slide covered with aluminum foil and air dried overnight. Samples were analyzed with a Renishaw inVia Raman spectrometer, equipped with a Leica optical microscope and a PRIOR automatic stage. The samples were irradiated with a 785 nm excitation laser and spectra were collected with a 50× long range objective in the range of 300-1800 cm-1. A 60 μm×60 μm area was mapped, to collect 25 spectra per sample.

Analytical Work

After collection, the data was imported into the MATLAB workspace. Here, spectral datasets for each sample were preprocessed for visualization and further data analysis. Initial preprocessing steps included assigning classes (race, gender, etc.), baseline correction using an air-PLS algorithm (Zhang et al., “Baseline Correction Using Adaptive Iteratively Reweighted Penalized Least Squares,” Analyst 135(5):1138-1146 (2010); which is hereby incorporated by reference in its entirety), removing spectra that exhibited cosmic rays, and interpolating axes so that all datasets have the same axis scale. Two calibration datasets were built for gender and race differentiation, using the preprocessed spectra, each containing 1,357 spectra.

After the initial preprocessing was completed, statistical modeling was carried out with the PLS Toolbox. Additional preprocessing steps, such as normalization and mean centering, were incorporated immediately before modeling. Two SVM-DA models were built for classification based on the two calibration datasets. Both models were internally cross-validated by Venetian blinds.

Results

Race Determination

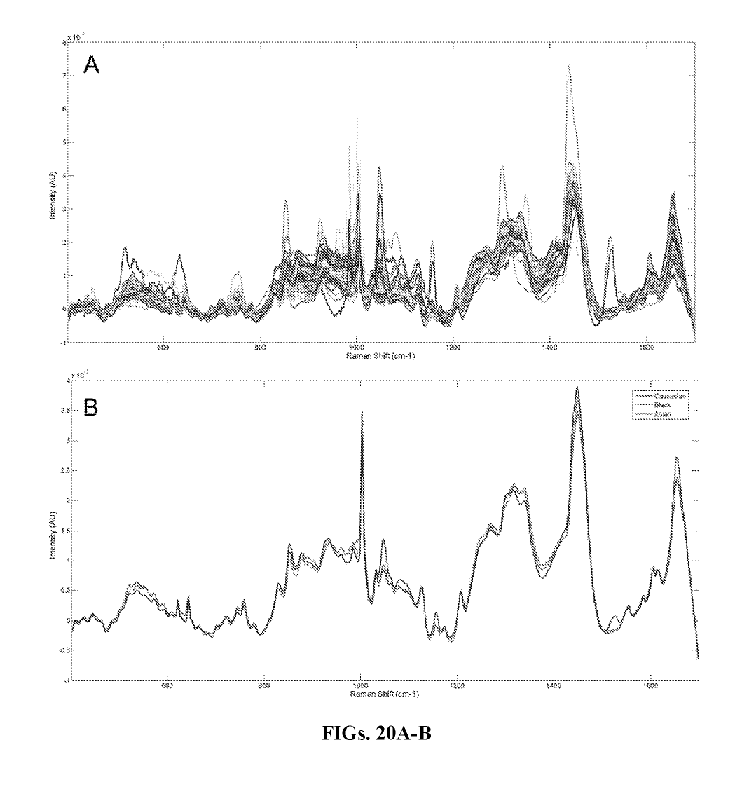

Saliva is a very heterogeneous body fluid, consisting of water, mucus, electrolytes, enzymes, and antibacterial compounds (Virkler et al., “Raman Spectroscopic Signature of Blood and its Potential Application to Forensic Body Fluid Identification,” Anal. Bioanal. Chem. 396(1):525-34 (2010), which is hereby incorporated by reference in its entirety). This complexity is reflected by the Raman spectra of saliva, showing contributions from several different chemical species. Glycoproteins from the mucus are made evident by the amide I peak at 1653 cm⁻¹ and the aromatic breathing peak at 1002 cm⁻¹. Low frequency peaks, 323-521 cm⁻¹, are due to polysaccharides. The averaged spectra for each donor and race can be seen in FIGS. 20A-B. While the spectra are visually similar, differences in relative peak intensity between races can be seen at 750, 878, 957, and 1654 cm⁻¹.

All of the collected spectra were combined to create the calibration dataset, from which an SVMDA model was built, with 10-fold cross validation splits. A confusion matrix, displaying the cross-validated predictions and accuracy, is shown in Table 8. A total of 469 spectra, out of 1,357, were misclassified. The overall accuracy of the model is 65.4%.

TABLE 8
Confusion Matrix Showing the Cross-Validated Results
of the Saliva Donor Race Differentiation SVM-DA Model.

| | Actual Class | | |
|---|---|---|---|
| Predicted Class (CV) | Caucasian | Black | Asian |
| Caucasian | 325 | 103 | 46 |
| Black | 100 | 316 | 158 |
| Asian | 22 | 40 | 247 |
| Accuracy | 73% | 69% | 55% |

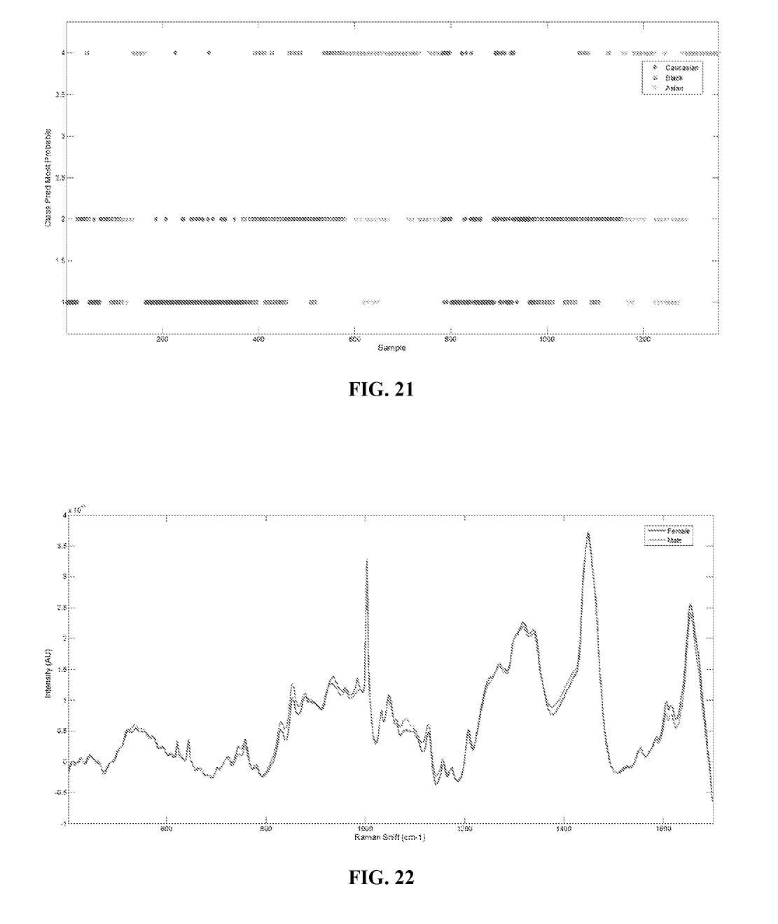

The class predictions are visualized two ways in FIG. 21. As a classification model, the SVMDA model assigns each spectrum to a single class. FIG. 21 shows these cross-validated class predictions for each spectrum in the calibration dataset.

Gender Determination

The ability to differentiate saliva samples according to donor gender was also investigated using chemometrics and the same dataset used for race differentiation. As described above, saliva is a heterogeneous body fluid and its Raman spectra indicated the presence of several biochemical components. The average Raman spectra from female and male donors are shown in FIG. 22. While the spectra are overall similar in peak position and intensity, there are visual differences between the two classes. For example, the three peaks between 913 and 933 cm⁻¹ are more pronounced and intense in the female spectrum than the male. However, the broad peak at 1314 cm⁻¹ is more intense in the male spectrum.

Just as with the race differentiation example, an SVMDA model was built using the calibration dataset, with 10-fold cross validation splits. Table 9 shows the confusion matrix for this calibration model. Out of all 1,357 spectra used to build the model, only 233 were misclassified. The cross-validated sensitivity and specificity rates of the model are 88.4% and 77.4%, respectively.

TABLE 9
Confusion Matrix Showing the Cross-Validated Results of
the Saliva Donor Gender Differentiation SVM-DA Model.

Actual Class

| Predicted Class | Female | Male |
|---|---|---|
| Female | 592 | 155 |
| Male | 78 | 532 |
| Accuracy | 88.4% | 77.4% |

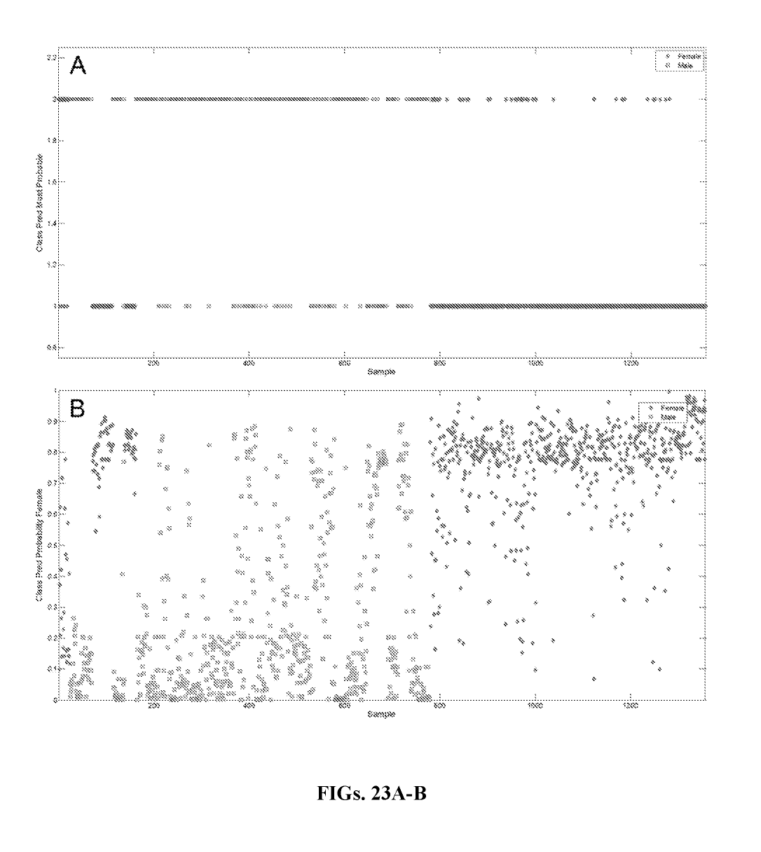

The prediction results of the model are shown in FIGS. 23A-B. The cross-validated class predictions made by the SVMDA model to differentiate the gender of saliva donors are shown in FIG. 23A. Each symbol represents a single Raman spectrum. Spectra from female donors should be located along the lower line, while spectra from male donors should be located along the upper line. Deviations from this pattern represent misclassifications. FIG. 23B shows the probability of each spectrum as being predicted as male. This plot illustrates that there is considerable confusion between the two genders on the part of the classification model.

### Conclusions

Two preliminary SVMDA models built on spectra collected from 60 saliva donors were constructed to differentiate donor gender and race. The donor population consisted of Caucasian, Black, and Asian donors, with an equal number of males and females. The average cross-validated accuracy of the preliminary race-based calibration model is 65.4%. The cross-validated sensitivity and specificity rates of the preliminary gender-based model are 77.4% and 88.4%, respectively. These results were all obtained through internal cross-validation. None of the models described above were submitted to external validation, a key step in method development.

### Example 12—Determine Race and Gender Based on Sweat

### Summary

This study looked into the potential to use Raman spectroscopy as a tool to determine an individual's race and gender using a sample of sweat. Raman spectra were collected from 20 sweat donors, and used to build two chemometric classification models. The cross-validated PLS-DA model built to differentiate race had an average sensitivity and specificity of 98.7 and 99.4%, respectively. The SVM-DA model that differentiated the genders of sweat donors had a 93.7% cross-validated sensitivity rate, and a 98.6% cross-validated specificity rate.

Experimental Work

A total of 20 sweat samples were purchased from Lee Biosolutions. The donor population consisted of 10 Caucasian, 7 Black, 2 Hispanic, and 1 Asian donor. The gender breakdown was 13 males, and 7 females. Sweat samples were prepared by depositing 10 μL onto an aluminum foil covered microscope slide, and allowed to dry overnight. Samples were analyzed via Raman mapping, with a 785 nm excitation laser and a 50× objective. Spectra were collected in the range of 300-1800 cm⁻¹, with three 10-second accumulations. Two mapping procedures were utilized. First, three areas on the sample were mapped, each containing 35 points/spectra, for a total of 105 spectra. In the interest of time efficiency, this was changed to one map consisting of 117 points/spectra. Because none of the irradiation, excitation, or collection parameters were altered, the spectral information obtained remained constant.

Analytical Work

Spectra were imported into MATLAB for preprocessing, and used to build models with the PLS Toolbox. First, spectra were assigned class labels, such as race, and gender. Next, spectra were truncated to reduce the spectral range to 500-1700 cm⁻¹. Lastly, spectra were filtered through PCA modeling to exclude outliers. A PCA model was constructed using all of the collected spectra, and those with high Hotelling T2 scores outside of the 95% confidence interval were excluded from the calibration dataset.

The preprocessed calibration dataset was then used to build chemometric models to differentiate the spectra on the basis of donor race or gender. Two SVM-DA calibration models were built. Final preprocessing steps executed during the model calibration phase included smoothing, normalization, and mean centering.

Results

Race Determination

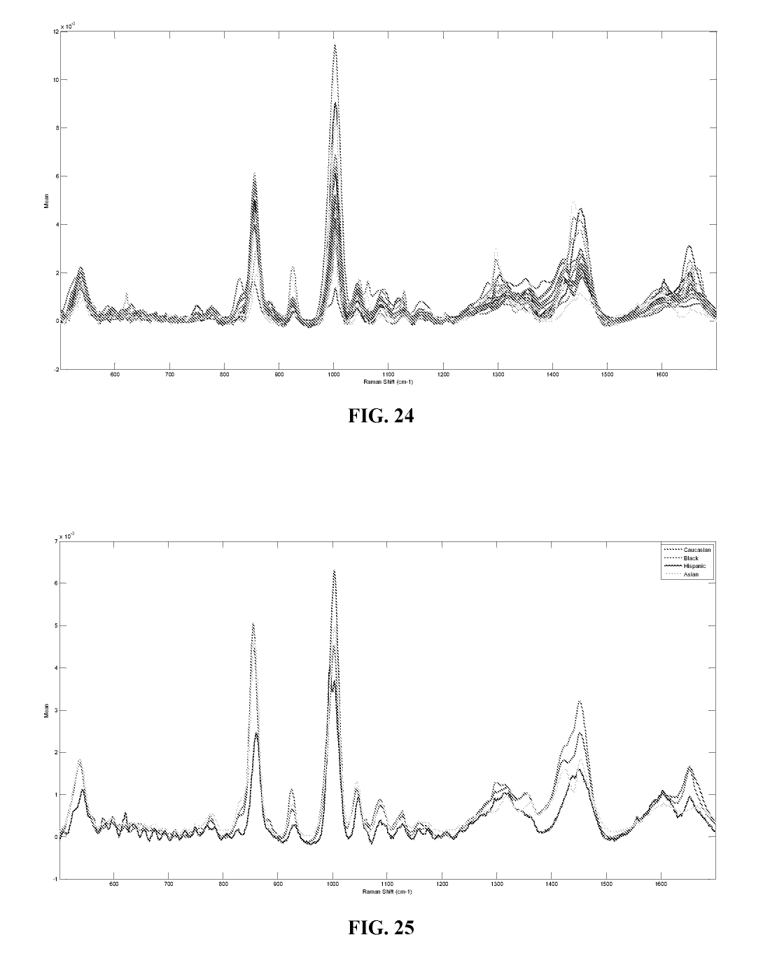

FIG. 24 shows the mean preprocessed Raman spectra for all 20 donors, while FIG. 25 shows the mean Raman spectra for each of the four races. Visible differences in spectral intensity are seen at 855 and 1003 cm⁻¹, which have been assigned to lactate and urea, respectively (Virkler et al., “Raman Spectroscopy Offers Great Potential for the Nondestructive Confirmatory Identification of Body Fluids,” Forensic Sci. Int. 181(1-3):e1-e5 (2008); Sikirzhytskaya et al., “Raman Spectroscopic Signature of Vaginal Fluid and its Potential Application in Forensic Body Fluid Identification,” Forensic Sci. Int. 216(1-3):44-8 (2012), which are hereby incorporated by reference in their entirety). Additionally, the region between the peaks at 1424 and 1452 cm⁻¹ show variations between the classes studied. These two peaks have also been attributed to urea and lactate (Virkler et al., “Raman Spectroscopy Offers Great Potential for the Nondestructive Confirmatory Identification of Body Fluids,” Forensic Sci. Int. 181(1-3):e1-e5 (2008); Sikirzhytskaya et al., “Raman Spectroscopic Signature of Vaginal Fluid and its Potential Application in Forensic Body Fluid Identification,” Forensic Sci. Int. 216(1-3):44-8 (2012), which are hereby incorporated by reference in their entirety).

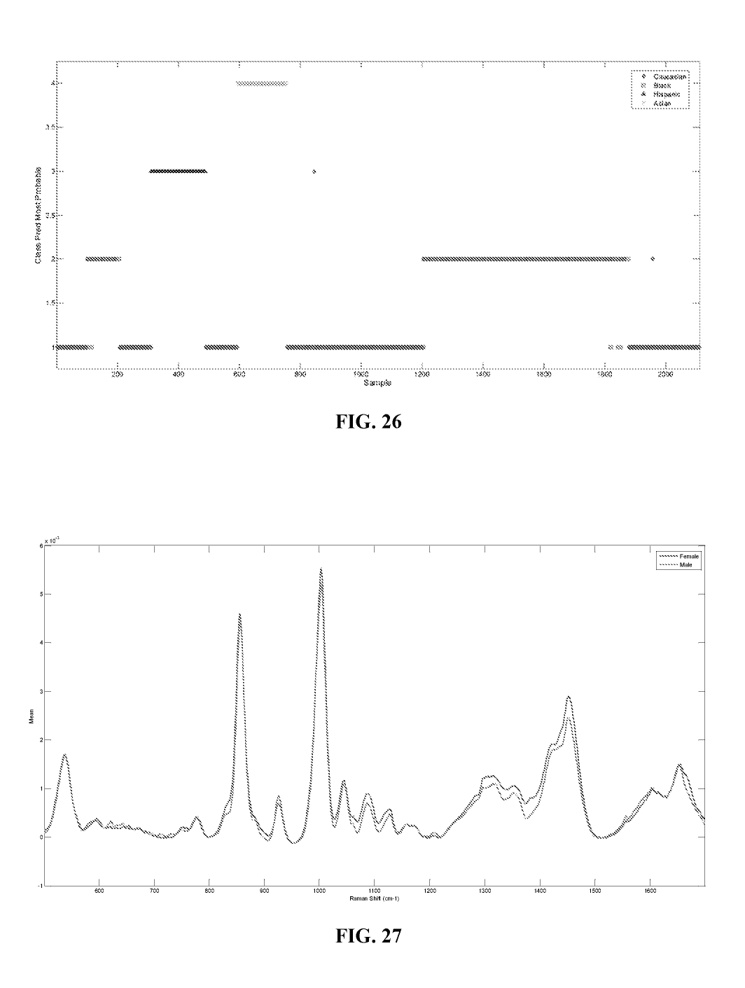

The first chemometric model constructed attempted to separate spectra from donors of differing races. Five latent variables were used to separate the four groups. FIG. 26 shows the most probable class predictions for this SVM-DA model, and the model's confusion matrix is shown in Table 10. The average cross-validated sensitivity for the model is 98.7%, and the average cross-validated specificity is 99.4%.

TABLE 10
Confusion Matrix Showing the Cross-Validated Results
of the Sweat Donor Race Differentiation SVM-DA Model.

| | |
|---|---|
| Predicted Class | Actual Class |

| (CV) | Caucasian | Black | Hispanic | Asian |
|---|---|---|---|---|
| Caucasian | 982 | 12 | 4 | 0 |
| Black | 7 | 762 | 0 | 0 |
| Hispanic | 2 | 1 | 174 | 0 |
| Asian | 3 | 2 | 0 | 162 |
| Accuracy | 98.8% | 98.1% | 97.8% | 100% |

Gender Determination

The same calibration dataset was used to build another SVM-DA model to differentiate donors by gender. FIG. 27 shows the mean preprocessed spectra for all 20 samples, colored by gender. When compared to FIG. 25, fewer deviations were observed between the classes. This indicates that the Raman characteristics of the different sample groups are more similar overall, and distinguishing between them may be more difficult than distinguishing between races.

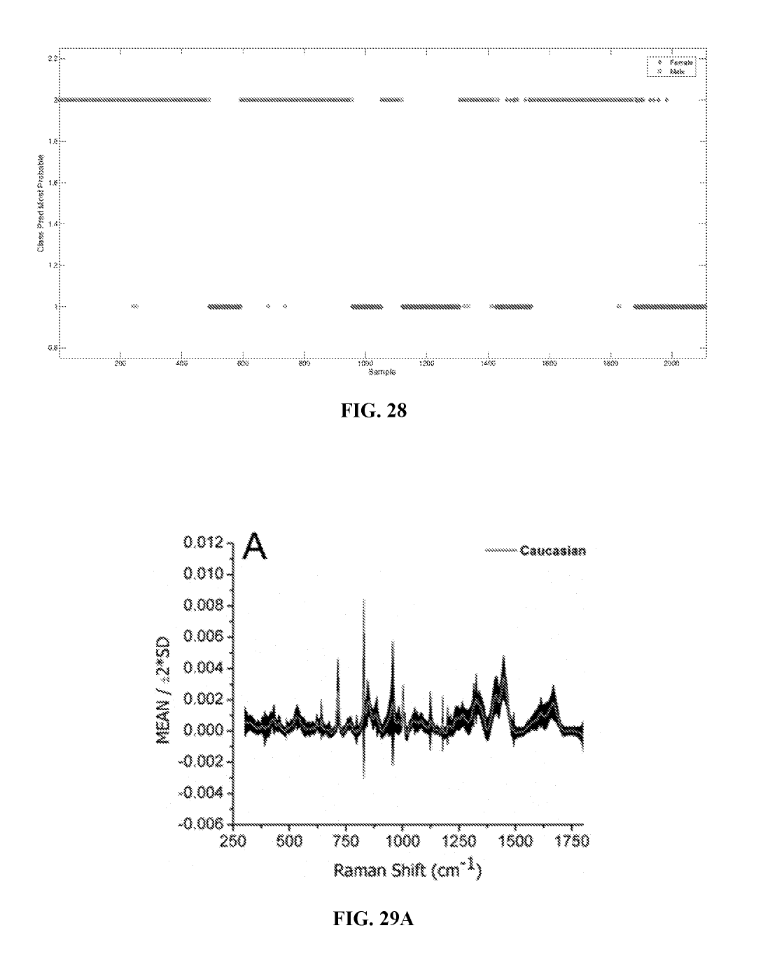

FIG. 28 illustrates the results from the internally cross-validated SVMDA gender differentiation model. Each symbol represents a single spectrum collected from a female (red diamond) or male (green square) donor. The cross validated sensitivity and specificity of this model are 93.7 and 98.6%, respectively. As expected, the classification error is higher than the race differentiation model. The confusion matrix for this model is displayed in Table 11.

TABLE 11
Confusion Matrix Showing the Cross-Validated Results
of the Sweat Donor Gender Differentiation SVM-DA Model.

Actual Class

| Predicted Class (CV) | Female | Male |
|---|---|---|
| Female | 697 | 19 |
| Male | 47 | 1348 |
| Accuracy | 93.7% | 98.6% |

### Conclusions

The present study sought to explore the potential to use Raman spectroscopy to identify a donor's race and gender using their sweat. The SVM-DA model built to differentiate race had an average cross-validated accuracy rate of 98.7%, while the SVM-DA model built to differentiate gender had an accuracy rate of 96.2%. The results reported in the present application do not include external validation of the models, a key step in method development.

### Example 13—Determine Race Based on Semen

### Summary

The main objective of this study was to develop a new method that can differentiate Raman spectra from dried semen traces based on the race of the donors. Raman spectra were acquired from human semen samples, from donors of three races (Caucasian, Black, and Hispanic). The spectra in the original dataset showed significant variation within and between donors, demonstrating semen's heterogeneous nature. Multivariate statistical analysis of Raman spectra was employed on the collected data to evaluate composition of semen samples, which varies with race. A PCA model was used to remove outliers (through Q residuals and Hotelling T2). ANN classification models reveal that the developed methodology has the definite potential to differentiate races.

Experimental Work

A total of 36 semen samples were acquired from Bioreclamation, LLC for this project. The population included 12 Caucasian, 12 Black, and 12 Hispanic donors. Samples were prepared by depositing 10 μL of semen onto an aluminum foil covered microscope slide and allowed to dry overnight. Samples were then analyzed the following day using a Renishaw inVia Raman spectrometer, equipped with a Leica microscope and PRIOR automatic stage. Data was collected by a 785 nm excitation laser in the range of 300-1800 cm⁻¹. Each semen sample was mapped to collect 64 spectra across a 2 mm² area, where each spectrum was the result of seven 10-second accumulations.

Analytical Work

The experimental spectra were imported into the MATLAB. All of the spectra were labeled according to donor number and race. The PLS Toolbox and R project were used for spectral preprocessing and modeling. Spectra were preprocessed by baseline correction using an airPLS algorithm. All spectra were normalized by total area and mean centered. PCA models were created for detecting outliers; spectra with the most abnormal Hotelling T² and Q residuals. The dataset was then split into training and test data according to the donors that were randomly selected for testing at the beginning of the statistical analysis. Because of the small sample size, the data could not be partitioned into similarly sized and large training and test data set. Thus, the challenge was to find a reasonable balance between training and test data set size. A slight increase in the prediction error for test data set might be acceptable in order to minimize the variability of the error estimate considered acceptable (to achieve a stable model). After careful consideration, the test dataset size was decided to be 3 donors. The training data was used to build three binary and one tertiary model for classification and discrimination between all three races using the ANN approach.

After creating a test dataset by moving spectra from three donors from a set of available data into an independent data set (never to be touched during cross-validation), the remaining dataset (33 donors in tertiary model, or 21 donors in binary models) was used to build the classification models. For unbiased assessment and to rule out the effect of the data set size, all four original training datasets were externally cross-validated. For each classification model, the original training datasets were randomly split into training (75%) and validating (25%) data subsets in 20 repetitions. The R Neuralnet package (Fritsch et al., “Neuralnet: Training of Neural Networks.” R package version 1.31 (2010), which is hereby incorporated by reference in its entirety) was used to design and train all models of artificial neural networks. Different network topologies have been tried in an attempt to find the optimum network architecture. Among them, the resilient backpropagation algorithm showed the best accuracy for the validation sets. Optimal network architecture was determined by varying the number of hidden layers and number of neurons in each layer between 10 and 600. For each classification model, its performance was reported and averaging was used to obtain an aggregate measure from these models. Thus, CV results are reported as the performance over all validation sets. Generalizability and predictive potential given by the external CV was additionally assessed by validation of the models with the test dataset containing the three donors that were set aside at the beginning of modeling. This step was used to make sure the model trained on the calibration data is generalizable and will correctly classify external, unknown, spectra.

Results

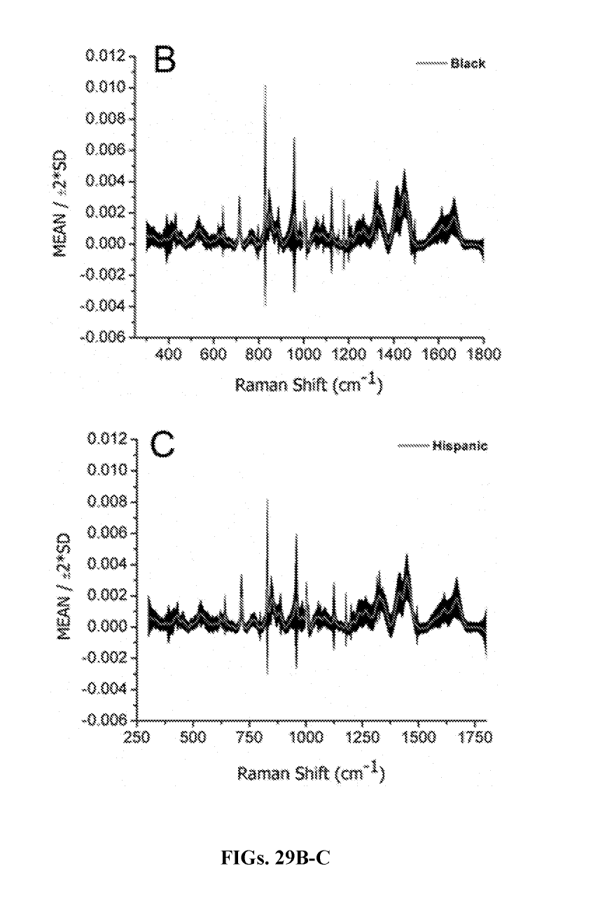

Despite the high heterogeneity observed both within and between donors, the mean spectra of semen from each group were found to be very similar with no significant spectral differences identified and they appear as typical characteristic bands for semen. FIGS. 29A-C show mean spectra for each group of subjects. The mean spectra are displayed along with +\− two standard deviations (SD) for the groups that were compared. Initial data set was reduced by eliminating outliers using PCA analysis.

The modeling process was carried out in six steps. First, the original dataset of 36 donors was divided into a training dataset of 33 donors, and a testing dataset of 3 donors. The test donors were set aside until the final step of validation. Second, the training dataset of 33 donors was divided further in an effort avoid overfitting and to build a robust ANN model. The training dataset was randomly split so that a bulk of the spectra (75%) was put into a training data subset, and the remaining spectra (25%) were put into a testing data subset. Third, the training data subset was used to calibrate an ANN model, which was then validated with the testing data subset. Steps 2 and 3 were repeated several times, each time with both a new random split and a new architecture scheme, until the ANN model parameters were optimized. Fourth, the “optimal” model architecture was cross-validated 20 times with new training and testing data subsets. The results from all 20 repetitions were recorded and used to make an average confusion matrix for the cross-validation phase. Fifth, the original training dataset, created in the first step, was used to train the final ANN model according to the optimal architecture scheme. The sixth and final step was external validation. The original testing dataset, containing the 3 donors set aside at the beginning, was used to externally validate the final ANN model.

In order to build binary models, all of the donors from one race were removed from the original dataset, and then all six modeling steps were carried out exactly as outlined above. This was done for three binary models, Caucasian vs. Black, Caucasian vs. Hispanic, and Black vs. Hispanic. A total of four final ANN models were built and externally validated.

The results from all four model's cross-validation phases are show in Table 12. During the cross-validation phase, the tertiary model achieved 89% accuracy in its predictions. For the binary models; the Caucasian vs. Black model achieved 96% accuracy, the Caucasian vs. Hispanic model achieved 94%, and the Black vs. Hispanic model achieved 91%.

TABLE 12
Confusion Matrices From all Four ANN
Model's Cross-Validation Phases.

| | Predicted | Actual Race | | |
|---|---|---|---|---|
| Model | Race | Caucasian | Black | Hispanic |
| Binary Model #1 | Caucasian | 163 | 8 | — |
| | Black | 6 | 138 | — |
| | Hispanic | — | — | — |
| Binary Model #2 | Caucasian | 155 | — | 10 |
| | Black | — | — | — |
| | Hispanic | 10 | — | 139 |
| Binary Model #3 | Caucasian | — | — | — |
| | Black | — | 134 | 14 |
| | Hispanic | — | 13 | 148 |
| Tertiary Model | Caucasian | 149 | 4 | 11 |
| | Black | 11 | 158 | 6 |
| | Hispanic | 10 | 12 | 153 |

The confusion matrices from all four model's external validation are reported in Table 13. After external validation, the tertiary model achieved 82% accuracy in its predictions. For the binary models, the Caucasian vs. Black model achieved 98% accuracy, the Caucasian vs. Hispanic model achieved 99%, and the Black vs. Hispanic model achieved 80%. A threshold of 50% was then used, such that if 50% or more of a particular donor's spectra are classified to a single race, the donor is ultimately classified to that race. Using this threshold, all three external validation donors were classified correctly by all four models.

TABLE 13
Confusion Matrices From All Four ANN
Model's External Validation Phases.

| | Predicted | Actual Race | | |
|---|---|---|---|---|
| Model | Race | Caucasian | Black | Hispanic |
| Binary Model #1 | Caucasian | 60 | 1 | — |
| | Black | 2 | 110 | — |
| | Hispanic | — | — | — |
| Binary Model #2 | Caucasian | 64 | — | 1 |
| | Black | — | — | — |
| | Hispanic | 0 | — | 123 |
| Binary Model #3 | Caucasian | — | — | — |
| | Black | — | 76 | 1 |
| | Hispanic | — | 36 | 63 |
| Tertiary Model | Caucasian | 34 | 0 | 1 |
| | Black | 4 | 68 | 14 |
| | Hispanic | 10 | 0 | 34 |

### Conclusions

Raman spectroscopy was used to analyze human semen samples and a new analytical approach was developed to determine a donor's race based on the spectroscopic data obtained. ANN models were used to classify each spectrum as belonging to one of the three races studied, Caucasian, Black, or Hispanic. After extensive cross-validation, the accuracy scores for three binary models, Caucasian vs. Black, Caucasian vs. Hispanic, and Black vs. Hispanic, were reported as 96%, 94%, and 91%, respectively. After external validation, these rates were 98%, 99%, and 80%. The tertiary model achieved an accuracy rate of 89% during cross-validation, and a rate of 82% during external validation. Finally, applying a threshold of 50% to the spectral predictions resulted in all three external validation donors being classified correctly. This was true for the tertiary model, as well as all three binary models.

### Example 14—Determine Race Based on Menstrual Blood

### Summary

The intention of this study is to develop a method capable of differentiating donor's races based on Raman spectra collected from dry human menstrual blood. All instrumental parameters were selected based on preliminary studies. PLS-DA and SVM-DA were chosen to construct simple classification models using a training dataset containing Raman spectra from five Caucasian and ten African American donors. One additional PLS-DA and SVM-DA model was built using only specific peaks selected by GA analysis. The number of components for each model was selected by choosing a local minimum of total data variance captured using a scree plot. All models were internally cross-validated and three of the four were externally validated.

Experimental Work

All menstrual blood samples were kept frozen until sample preparation. For each blood sample, approximately 10 μL was placed on an aluminum covered microscope slide and allowed to dry overnight prior to analysis. A Renishaw inVia confocal Raman spectrometer and a PRIOR automatic stage were used for data collection for all menstrual blood samples. The instrument was calibrated with a silicon standard before all measurements. Spectra were accumulated with a 20× long working distance objective and 785 nm excitation laser in the spectral range of 300-1800 cm⁻¹. Laser power at the sample was approximately 4.0 mW. A Raman map consisting of 15 spectra were collected from each of the samples. WiRE software version 3.2 was used to operate the instrument.

Analytical Work

All data preparation and construction of statistical models were performed with the PLS Toolbox 7.5.3 (Eigenvector Research, Inc., Wenatchee, Wash.) operating in MATLAB and Statistics Toolbox Release R2012b (Mathworks, Inc., Natick, Mass.). For each sample, the 15 spectra were smoothed with a second-order polynomial and filter width of 15, baseline corrected with a 6th order polynomial, and normalized by total area. After the preprocessing steps, the spectra were mean centered before models were calculated.

In order to eliminate the non-informative and redundant variables from the datasets, GA was applied, which is an evolutionary feature selection method. GA considers all of the variables within a Raman spectral dataset and their significance, or contribution, to the discrimination process. This allows for a reduction of the original Raman spectra to a smaller subset(s) of wavenumbers in order to improve prediction performance. The technique is especially helpful in cases when the spectral dataset consists of hundreds or thousands variables. A detailed explanation of GA for variable selection and its applications was published by Niazi and Leardi (Niazi et al., “Genetic Algorithms in Chemometrics,” Journal of Chemometrics 26(6):345-351 (2012), which is hereby incorporated by reference in its entirety). The population size was set to 72, the maximum number of generations was set to 100, the breeding crossover rule was set to double crossover, and the default mutation rate was used (0.005). Finally, a total of 200 runs were performed.

Two PLS-DA models were constructed, one for 214 preprocessed spectra (11 outliers removed) and another using the genetic algorithm selected peaks for all 225 spectra. Two SVM-DA models were also constructed, one using the 225 total spectra and the other using the genetic algorithm selected peaks for all 225 spectra. All models were internally cross-validated using the venetian blinds method and three were externally cross-validated using a donor-wise leave-one-out approach.

Results

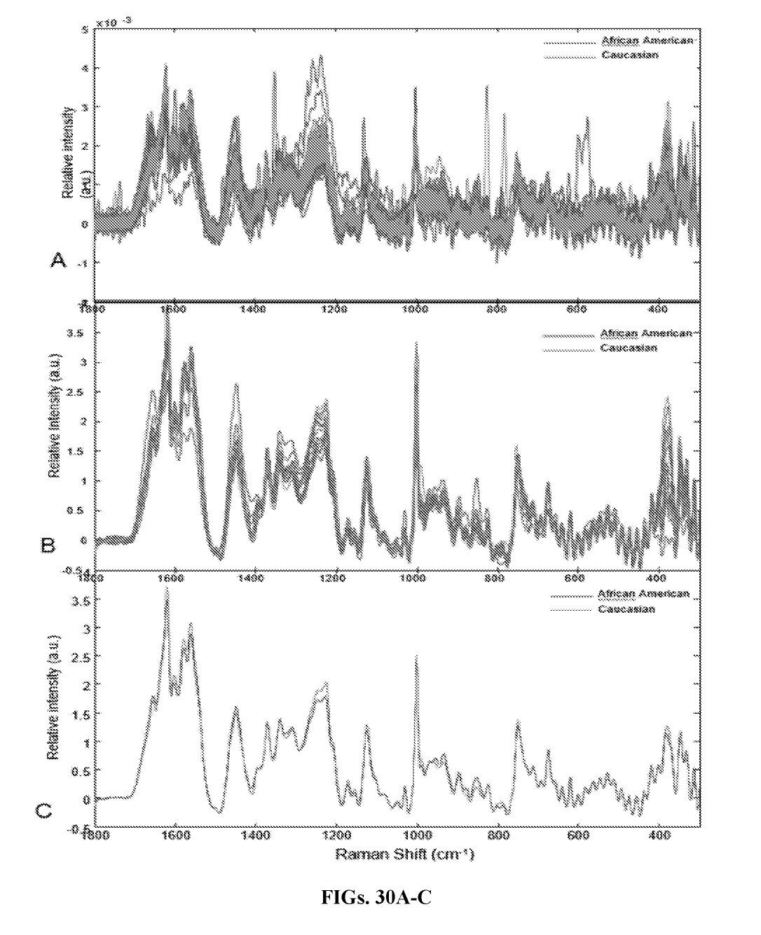

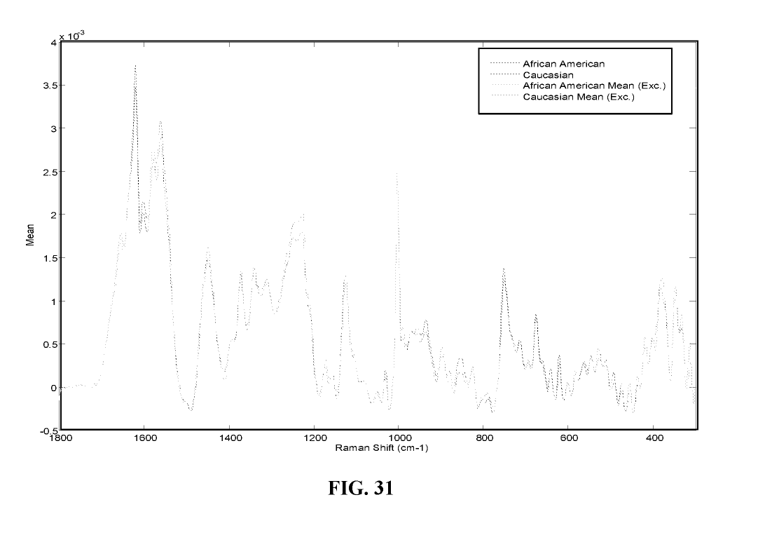

For each menstrual blood sample, a Raman spectral map of 15 points was collected. The spectra were preprocessed by smoothing, baseline correction and normalization by total area. They were also averaged by donor and race to study the differences within the peaks. The training dataset for the first PLS-DA model consisted of 214 preprocessed spectra. The 225 total preprocessed spectra used for model building are shown in FIG. 30A. From visual inspection of FIG. 30B (averaged by donor) and FIG. 30C (averaged by race), all spectra look to be identical in terms of the number of spectral features and their location. However, variations in the relative intensity of several peaks are noticeable. GA analysis was used to determine which peaks were more informative for race discrimination. FIG. 31 shows the averaged menstrual blood spectrum for both races with the GA selected peaks as a darker shade of red (African American) or green (Caucasian).

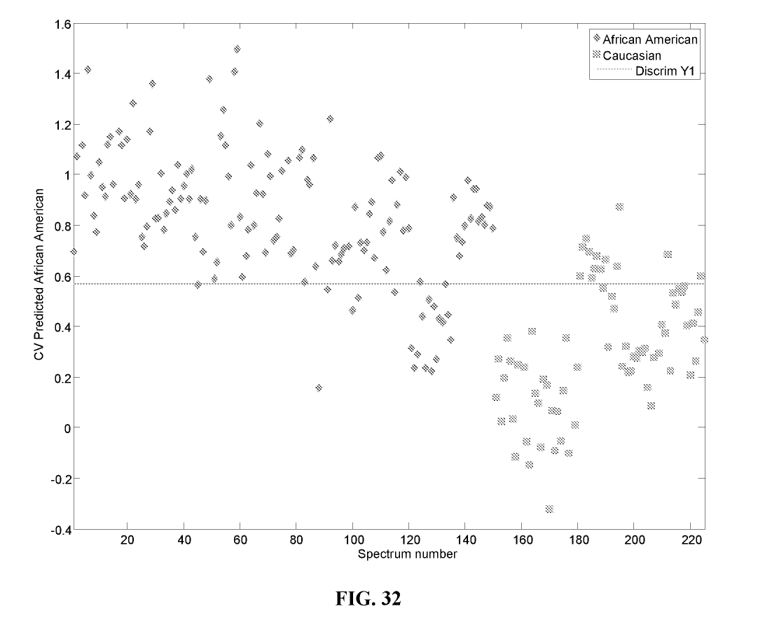

The first PLS-DA model was constructed using a training dataset containing only 214 of the 225 total preprocessed spectra. Eleven of the 225 spectra were outside the 95% confidence interval on the Hotelling T² and Q Residuals scores plot and were removed from the original training dataset to improve the results. The model was built using four LVs. The cross-validated prediction results for the African American class for the first PLS-DA model can be seen in FIG. 32. This plot displays the prediction scores for each spectrum in the training dataset after internal cross-validation. Any symbol (spectrum) that lies above the threshold (red line) would be predicted as belonging to the African American class. Table 14 shows the number of correctly and incorrectly classified spectra for this PLS-DA model. The sensitivity and specificity values for the African American class were 0.859 and 0.819, respectively, and vice-versa for the Caucasian class.

TABLE 14
Confusion Table for Cross-Validated Prediction Results
of the First PLS-DA Model (Built with 214 Spectra).

Predicted Class

| Actual Class | African American | Caucasian |
|---|---|---|
| African American | 122 | 20 |
| Caucasian | 13 | 59 |

An external validation was made for the model by taking out one single donor, rebuilding the model and making predictions for the donor removed. All donors were removed one by one and the model was rebuilt each time. The true positive (TP) and false negative (FN) results for race predictions are displayed in Table 15. The average TP and FN values for the donor-wise external validation for the first PLS-DA model (built with 214 spectra) were 0.64 and 0.37 for the African American class and 0.33 and 0.67 for the Caucasian class, respectively.

TABLE 15
Results for Donor-Wise External Validation of the
First PLS-DA Model (Built with 214 Spectra).

| | | Predicted Class | | | |
|---|---|---|---|---|---|
| | | African | | True | False |
| Donor | Actual Class | American | Caucasian | Positive | Negative |
| 1 | African American | 14 | 0 | 1.00 | 0.00 |
| 2 | African American | 14 | 0 | 1.00 | 0.00 |
| 3 | African American | 13 | 2 | 0.87 | 0.13 |
| 4 | African American | 12 | 2 | 0.86 | 0.14 |
| 5 | African American | 15 | 0 | 1.00 | 0.00 |
| 6 | African American | 6 | 5 | 0.55 | 0.45 |
| 7 | African American | 1 | 13 | 0.07 | 0.93 |
| 8 | African American | 11 | 4 | 0.73 | 0.27 |
| 9 | African American | 0 | 15 | 0.00 | 1.00 |
| 10 | African American | 4 | 11 | 0.27 | 0.73 |
| 11 | Caucasian | 5 | 9 | 0.64 | 0.36 |
| 12 | Caucasian | 14 | 0 | 0.00 | 1.00 |
| 13 | Caucasian | 15 | 0 | 0.00 | 1.00 |
| 14 | Caucasian | 2 | 12 | 0.86 | 0.14 |
| 15 | Caucasian | 13 | 2 | 0.13 | 0.87 |

The second PLS-DA model was constructed using the GA selected peaks. The model was built using two LVs. The cross-validated prediction results for the African American class for the second PLS-DA model can be seen in FIG. 32. Any symbol (spectrum) that lies above the threshold (red line) would be predicted as belonging to the African American class. Table 16 shows the number of correctly and incorrectly classified spectra for this PLS-DA model. The sensitivity and specificity values for the African American class were 0.547 and 0.993, respectively, and vice-versa for the Caucasian class. The results for this model, built using the GA selected peaks demonstrated much worse results for internal cross-validation predictions than the first PLS-DA model constructed. Based on this observation, it was decided not to perform a donor-wise external validation for the second PLS-DA model (built with using the GA selected peaks).

TABLE 16
Confusion Table For Cross-Validated Prediction Results of the
Second PLS-DA Model (Built Genetic Algorithm Selected Peaks).

Predicted Class

| Actual Class | African American | Caucasian |
|---|---|---|
| African American | 82 | 68 |
| Caucasian | 5 | 70 |

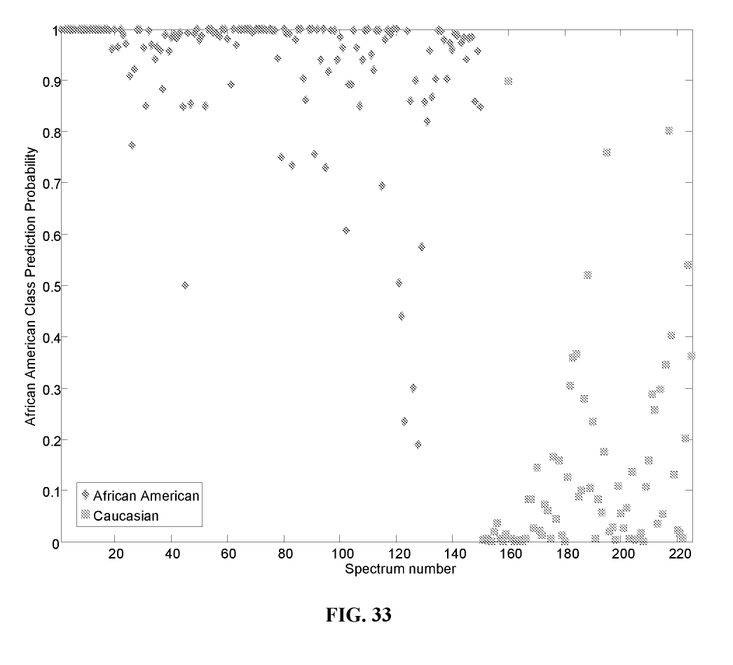

The first SVM-DA model was constructed using a training dataset containing 225 preprocessed spectra. The model was built using two LVs. The African American class prediction probability plot for this model can be seen in FIG. 33. This plot displays the spread of the spectra between the two races. A value of 1 represents a classification as African American and a value of zero represents a classification as Caucasian. Table 17 shows the number of correctly and incorrectly classified spectra, under cross-validation, for this SVM-DA model. The sensitivity and specificity values for the African American class were 0.867 and 0.787, respectively, and vice-versa for the Caucasian class.

TABLE 17
Confusion Table for Cross-Validated Results of the First
SVM-DA Model Built with 225 Spectra

Predicted Class

| Actual Class | African American | Caucasian |
|---|---|---|
| African American | 130 | 20 |
| Caucasian | 16 | 59 |

An external validation was carried out for the first SVM-DA model using the same principle described above for the PLS-DA model. The results for race predictions, TP and FN assignments are displayed in Table 18. The average TP and FN values for the donor-wise external validation for the first SVM-DA model were 0.69 and 0.31 for the African American class, and 0.28 and 0.72 for the Caucasian class, respectively.

TABLE 18
Results For Donor-Wise External Validation of the
First SVM-DA Model Built with 225 Spectra.

| | Actual | Predicted Class | | True | False |
|---|---|---|---|---|---|
| Donor | Class | African American | Caucasian | Positive | Negative |
| 1 | African | 15 | 0 | 1.00 | 0.00 |
| | American | | | | |
| 2 | African | 14 | 1 | 0.93 | 0.07 |
| | American | | | | |
| 3 | African | 14 | 1 | 0.93 | 0.07 |
| | American | | | | |
| 4 | African | 13 | 2 | 0.87 | 0.13 |
| | American | | | | |
| 5 | African | 15 | 0 | 1.00 | 0.00 |
| | American | | | | |
| 6 | African | 7 | 8 | 0.47 | 0.53 |
| | American | | | | |
| 7 | African | 1 | 14 | 0.07 | 0.93 |
| | American | | | | |
| 8 | African | 11 | 4 | 0.79 | 0.27 |
| | American | | | | |
| 9 | African | 0 | 15 | 0.00 | 1.00 |
| | American | | | | |
| 10 | African | 13 | 2 | 0.87 | 0.13 |
| | American | | | | |
| 11 | Caucasian | 4 | 11 | 0.73 | 0.27 |
| 12 | Caucasian | 15 | 0 | 0.00 | 1.00 |
| 13 | Caucasian | 15 | 0 | 0.00 | 1.00 |
| 14 | Caucasian | 5 | 10 | 0.67 | 0.33 |
| 15 | Caucasian | 15 | 0 | 0.00 | 1.00 |

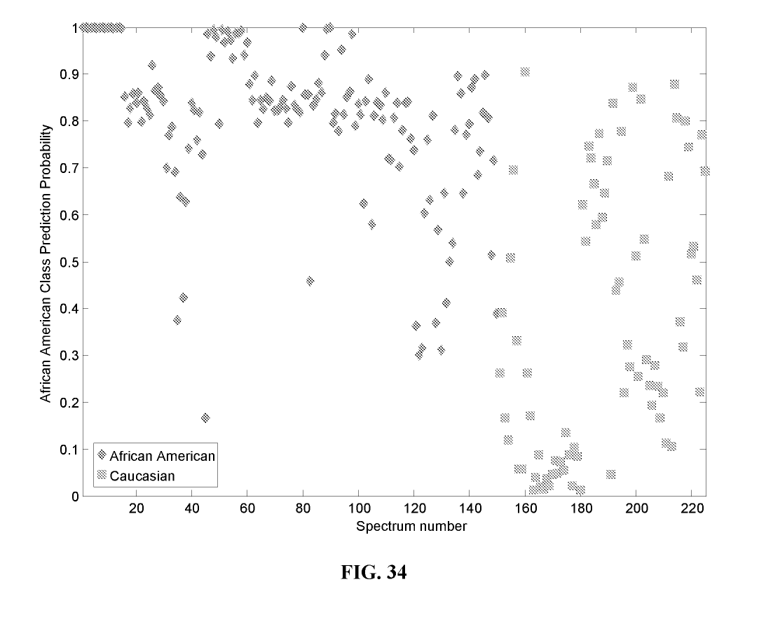

The second SVM-DA model was constructed using only the specific peaks selected by the GA analysis. The model was built using two LVs. The African American class prediction probability plot for this model can be seen in FIG. 34. A value of 1 represents a classification as African American and a value of zero represents a classification as Caucasian. Table 19 shows the number of correctly and incorrectly classified spectra, under cross-validation, for the second SVM-DA model built (using only the GA selected peaks). The sensitivity and specificity values for the African American class were 0.907 and 0.587, respectively, and vice-versa for the Caucasian class. An external validation was also performed for this SVM-DA model. The results for race predictions, TP and FN are displayed in Table 20. The average TP and FN values for the donor-wise external validation for the first SVM-DA model were 0.73 and 0.28 for the African American class, and 0.07 and 0.93 for the Caucasian class, respectively.

TABLE 19
Confusion Table for Cross-Validated Results of
SVM-DA Model Built Genetic Algorithm Selected Peaks.

Predicted Class

| Actual Class | African American | Caucasian |
|---|---|---|
| African American | 136 | 14 |
| Caucasian | 31 | 44 |

TABLE 20
Results for Donor-Wise External Validation of SVM-DA
Model Built With Genetic Algorithm Selected Peaks.

| | | Predicted Class | | | |
|---|---|---|---|---|---|
| | | African | | True | False |
| Donor | Actual Class | American | Caucasian | Positive | Negative |
| 1 | African | 15 | 0 | 1.00 | 0.00 |
| | American | | | | |
| 2 | African | 15 | 0 | 1.00 | 0.00 |
| | American | | | | |
| 3 | African | 8 | 7 | 0.53 | 0.47 |
| | American | | | | |
| 4 | African | 14 | 1 | 0.93 | 0.07 |
| | American | | | | |
| 5 | African | 15 | 0 | 1.00 | 0.00 |
| | American | | | | |
| 6 | African | 14 | 1 | 0.93 | 0.07 |
| | American | | | | |
| 7 | African | 11 | 4 | 0.73 | 0.27 |
| | American | | | | |
| 8 | African | 13 | 2 | 0.87 | 0.13 |
| | American | | | | |
| 9 | African | 2 | 13 | 0.13 | 0.87 |
| | American | | | | |
| 10 | African | 2 | 13 | 0.13 | 0.87 |
| | American | | | | |
| 11 | Caucasian | 10 | 5 | 0.33 | 0.67 |
| 12 | Caucasian | 15 | 0 | 0.00 | 1.00 |
| 13 | Caucasian | 15 | 0 | 0.00 | 1.00 |
| 14 | Caucasian | 15 | 0 | 0.00 | 1.00 |
| 15 | Caucasian | 15 | 0 | 0.00 | 1.00 |

### Conclusions

Four different statistical models were constructed using a training dataset of Raman spectral data collected from menstrual blood from ten African American donors and five Caucasian donors. The models constructed with the entire spectral range showed better internal classification when compared to the models constructed using the GA selected peaks. Furthermore, the models were tested via external validation of individual donors, which were excluded from the training dataset one by one. The PLS-DA model built with GA selected peaks was not subjected to the external validation because it did not show promising results for the internal classification. The results obtained for the external validation of the PLS-DA and SVM-DA models constructed with all preprocessed spectra were similar to each other. However, the PLS-DA model showed better sensitivity and specificity for the Caucasian class while the SVM-DA model showed better results for the African American class. With the number of samples analyzed, and the parameters chosen for using Raman spectroscopy combined with statistical modeling, it was not possible to sufficiently differentiate between menstrual blood from African American and Caucasian donors.

### Example 15—Determine Race and Gender Based on Dry Blood Traces Using ATR-FTIR Spectroscopy

### Summary

ATR-FTIR spectroscopy was applied to distinguish between genders and races from human blood. The sample collection included donors of both genders, and Caucasian, Black and Hispanic races. A calibration dataset of thirty donors was used to build models. The final SVM-DA models show donors' classification with 87% accuracy for each group respectively.

Experimental Work

The examination was performed on blood collected from 30 donors in total. The collection included 16 males and 14 females with 10 donors per race. For all the blood samples, a 20 μL drop was deposited onto a microscope slide and allowed to dry overnight. Each sample was scraped off from the glass slide and placed onto the instrument's crystal for data collection. A PerkinElmer Spectrum 100 FT-IR spectrometer with a diamond/ZnSe crystal was used for analysis. Spectra were recorded in the range 600-4000 cm⁻¹ with a spectral resolution of 4 cm⁻¹. Prior to placing the sample on the crystal for each measurement a background check was performed. Samples were scanned 10 times, with 32 accumulations per spectrum.

Analytical Work

For data treatment and advanced statistical analyses R (The R project. “R: A language and environment for statistical computing,” R Foundation for Statistical Computing, Vienna, Austria, ISBN 3-900051-07-0, [Available from: www.R project.org] (February 2016): package pROC, Robin X., et al., “pROC: An Open-Source Package for R and S+ to Analyze and Compare ROC Curves,” BMC Bioinformatics 12(1):77 (2011)) and MATLAB software (MATLAB and Statistics Toolbox Release R2012b (Mathworks, Inc., Natick, Mass.)) were used. For all 300 collected spectra, transmission to absorbance (log (1/T)), 2nd order derivative, normalization by total area and mean centering were applied for pretreatment. After these necessary preprocessing procedures statistical analysis was performed using the PLS Toolbox 7.5.3 (Eigenvector Research, Inc., Wenatchee, Wash.). Spectral fingerprint regions were identified via GA analysis. PCA and SVM-DA were used to distinguish the race and gender of different donors. After necessary preprocessing steps, multivariate outlier removal was carried out through PCA.

Results

In order to optimize the prediction results of SVM, the GA was again used to progressively reduce the wavenumber selection and the number of latent variables to be included. The population size was set to 70, the maximum number of generations was set to 100, the breeding crossover rule was set to double crossover, and the default mutation rate was used (0.005). Finally, a total of 100 runs were performed.

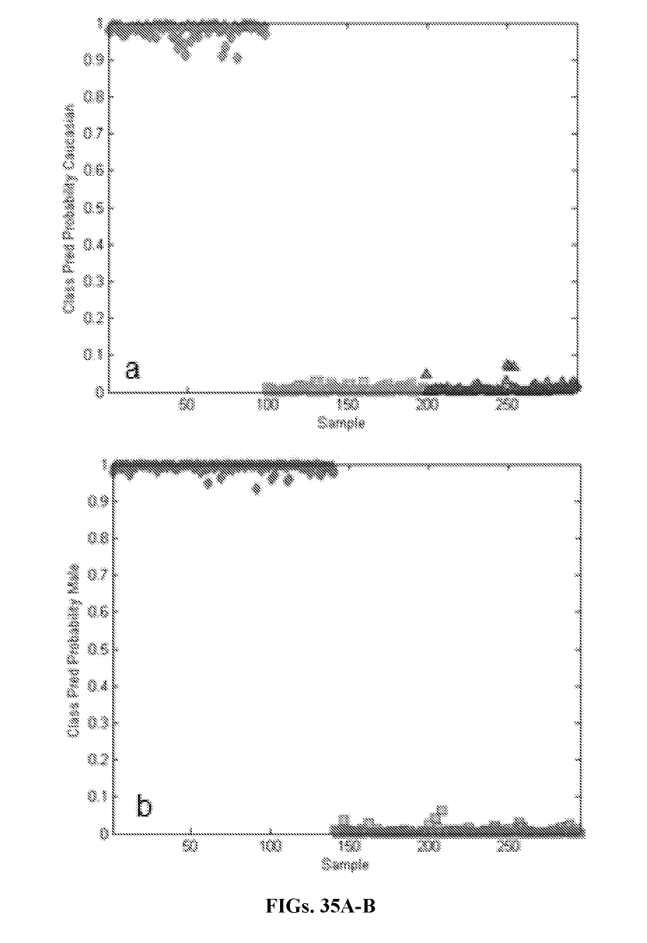

SVM modeling was applied to distinguish between races using the input features selected by GA from original infrared spectra. For this study Radial Basis Function (RBF) as a kernel function was optimized by a combined approach of Venetian blind cross-validation (five samples out) and a systematic grid search of the parameters. To evaluate the subject-independent accuracy performance of the SVM-DA models, all data from all subjects were divided subject-wise, so that spectra from one subject was placed aside from training set and served as a test set. The model was refit to each training set and validated in a blind manner on the corresponding test set. Validation results are reported as the average performance over all test sets. Among all 30 subjects, the probabilities for each spectrum and subject to belong to each class were recorded. ROC curves and AUC values were computed using SVM models to estimate the discriminatory power. Note that in the case of race differentiation, ROC analysis produced three ROC curves, one for each of the three classes compared to the others by binary models. FIGS. 35A-B show SVM-DA results for race (FIG. 35A) and gender (FIG. 35B) spectra binary calibration model (training stage). Note that the actual result of classification depends on threshold values which can be arbitrarily set to specific values.

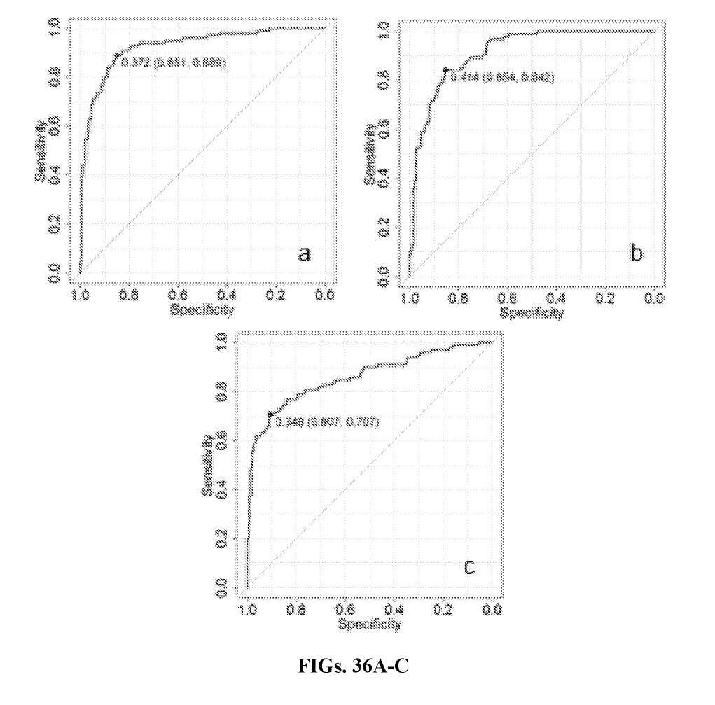

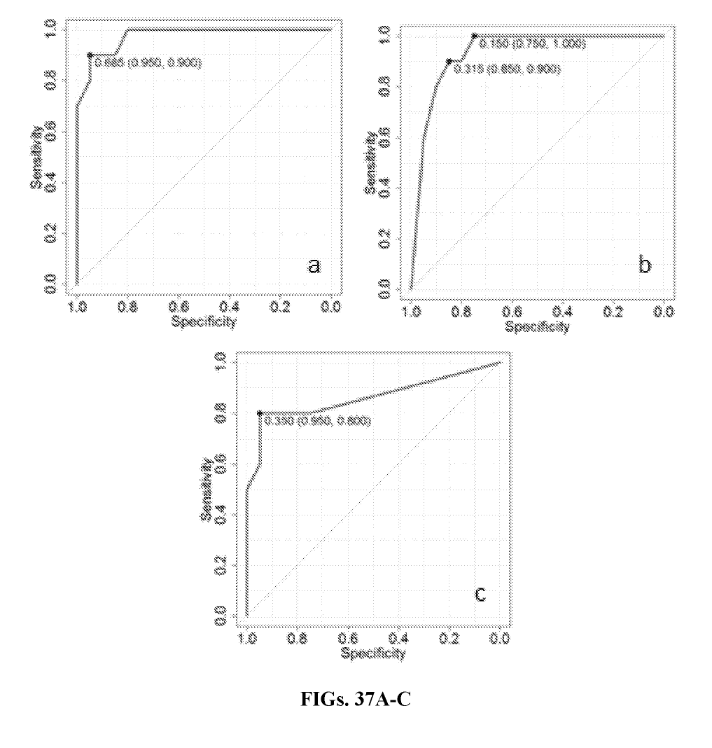

The principal of ROC analysis was used to assess the diagnostic accuracy of the SVM models in external donor-wise cross-validation. The AUCs of ROC curves were estimated by the trapezoidal method of integration with the corresponding 95% CI that have been evaluated with the method described by DeLong et al. (DeLong et al., “Comparing the Areas under Two or More Correlated Receiver Operating Characteristic Curves: A Nonparametric Approach,” Biometrics 837-845 (1988), which is hereby incorporated by reference in its entirety). Results of ROC analysis for race differentiation are depicted in FIGS. 36A-C for individual spectra and in FIGS. 37A-C for individual subjects, where the AUCs report probability for all classifiers that randomly chosen spectra will be correctly classified. Regarding the AUCs for race classification, the highest classification performance was accomplished with prediction of the Caucasian race based on individual spectra (AUC=0.93; 95% CI: 0.89-0.96) and on individual donors (AUC=0.98; 95% CI: 0.93-1.00). Also other SVM classifiers exhibit high performance. Using FTIR spectra enabled discrimination of the Hispanic race with AUC=0.91 (95% CI: 0.88-0.95) for individual spectra, AUC=0.94 (95% CI: 0.85-1.00) for individual donors and discrimination of the Black race with AUC=0.86 (95% CI: 0.81-0.91) for individual spectra, and AUC=0.86 (95% CI: 0.70-1.00) for individual donors.

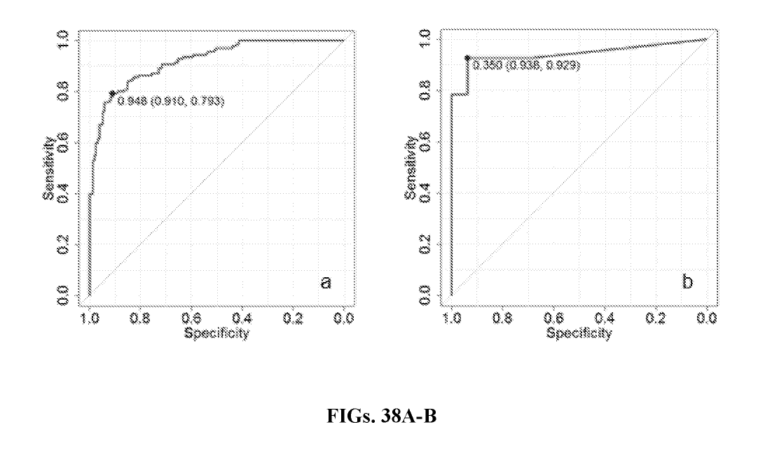

FIGS. 38A-B depict similar plots as in FIGS. 36A-C and 37A-C, showing ROC evaluation for the prediction of gender from FTIR spectra. This presents the ROC curves of the SVM models for external donor-wise cross-validation where only two classes are considered, i.e. male and female. Using FTIR spectra enabled discrimination of gender with the AUC of 0.92 (95% CI: 0.89-0.95) and 0.94 (95% CI: 0.85-1.00) based on a single spectrum and each subject respectively.

### Conclusions

A new technique has been applied to discriminate race and gender from human blood traces. ATR-FTIR with chemometrics has successfully distinguished between donors. Based on the two models that were built for gender and race differentiation, 26 of the 30 donors were classified correctly. Statistical parameters, as well as sensitivity and specificity values, were calculated for each model. The initial results show promise and validation testing is underway. This study demonstrates a great potential of FTIR spectroscopy combined with advanced statistics for forensic analysis of biological stains. To strengthen the results and validate the models, a blind test with unknown blood samples should be performed and is a future approach for this experiment.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of identifying gender and/or race of a subject using a body fluid stain from the subject, said method comprising:
providing a sample containing a body fluid stain from the subject;
providing a statistical model for determination of gender and/or race of a subject;
subjecting the sample or an area of the sample containing the stain to a spectroscopic analysis to produce a spectroscopic signature for the sample; and
applying the spectroscopic signature for the sample to the statistical model to ascertain gender and/or race of the subject, wherein the body fluid is selected from the group consisting of saliva, sweat, urine, semen, and vaginal fluid.

2.-3. (canceled)

4. The method of claim 1, wherein said applying determines the gender of the subject.

5. The method of claim 1, wherein said applying determines the race of the subject.

6. The method of claim 5, wherein said method determines the race of the subject as being black, white, asian, or hispanic.

7. (canceled)

8. The method of claim 1, wherein spectroscopic analysis is selected from the group consisting of Raman spectroscopy, mass spectrometry, fluorescence spectroscopy, laser induced breakdown spectroscopy, infrared spectroscopy, scanning electron microscopy, X-ray diffraction spectroscopy, powder diffraction spectroscopy, X-ray luminescence spectroscopy, inductively coupled plasma mass spectrometry, capillary electrophoresis, and atomic absorption spectroscopy.

9. (canceled)

10. The method of claim 8, wherein the Raman spectroscopy is selected from the group consisting of resonance Raman spectroscopy, normal Raman spectroscopy, Raman microscopy, Raman microspectroscopy, NIR Raman spectroscopy, surface enhanced Raman spectroscopy (SERS), tip enhanced Raman spectroscopy (TERS), Coherent anti-Stokes Raman scattering (CARS), and Coherent anti-Stokes Raman scattering microscopy.

11. (canceled)

12. The method of claim 8, wherein the Infrared spectroscopy is selected from the group consisting of Infrared microscopy, Infrared microspectroscopy, Infrared reflection spectroscopy, Infrared absorption spectroscopy, attenuated total reflection infrared spectroscopy, Fourier transform infrared spectroscopy, and attenuated total reflection Fourier transform infrared spectroscopy.

13. (canceled)

14. The method of claim 1, wherein the statistical model for determination of gender and/or race of a subject is prepared by multivariate analysis.

15. (canceled)

16. The method of claim 1, wherein the statistical model is prepared by classification statistical analysis.

17. The method of claim 16, wherein the classification statistical analysis is selected from the group consisting of Partial least squares discriminant analysis (PLS-DA), Support vector machines discriminant analysis (SVMDA), K-Nearest neighbor (KNN), Artificial neural network (ANN), and Soft independent modeling of/by class analogy (SIMCA).

18. A method of establishing a statistical model for determination of gender and/or race of a subject using a body fluid stain from the subject, said method comprising:
providing a plurality of samples containing a known type of body fluid stain from a subject of known race and/or gender;
subjecting each sample or an area of each sample containing the stain to a spectroscopic analysis to produce a spectroscopic signature for each sample; and
establishing a statistical model for determination of gender and/or race of a subject for a particular body fluid type based on said subjecting, wherein the body fluid is selected from the group consisting of saliva, sweat, urine, semen, and vaginal fluid.

19.-21. (canceled)

22. The method of claim 18, wherein the statistical model for determination of gender and/or race of a subject is prepared by multivariate analysis.

23. (canceled)

24. The method of claim 18, wherein the statistical model is prepared by classification statistical analysis.

25. (canceled)

26. The method of claim 18 further comprising
rebuilding the statistical model; and
validating the statistical model.

27. The method of claim 18 further comprising
performing an informative spectral features selection for further developing a spectroscopic signature.

28. The method of claim 18, wherein said establishing produces a statistical model for determination of the subject's gender for a specific type of body fluid.

29. The method of claim 18, wherein said establishing produces a statistical model for determination of the subject's race for a specific type of body fluid.

30. The method of claim 18, wherein spectroscopic analysis is selected from the group consisting of Raman spectroscopy, mass spectrometry, fluorescence spectroscopy, laser induced breakdown spectroscopy, infrared spectroscopy, scanning electron microscopy, X-ray diffraction spectroscopy, powder diffraction spectroscopy, X-ray luminescence spectroscopy, inductively coupled plasma mass spectrometry, capillary electrophoresis, and atomic absorption spectroscopy.

31. (canceled)

32. The method of claim 30, wherein the Raman spectroscopy is selected from the group consisting of resonance Raman spectroscopy, normal Raman spectroscopy, Raman microscopy, Raman microspectroscopy, NIR Raman spectroscopy, surface enhanced Raman spectroscopy (SERS), tip enhanced Raman spectroscopy (TERS), Coherent anti-Stokes Raman scattering (CARS), and Coherent anti-Stokes Raman scattering microscopy.

33. (canceled)

34. The method of claim 30, wherein the Infrared spectroscopy is selected from the group consisting of Infrared microscopy, Infrared microspectroscopy, Infrared reflection spectroscopy, Infrared absorption spectroscopy, attenuated total reflection infrared spectroscopy, Fourier transform infrared spectroscopy, and attenuated total reflection Fourier transform infrared spectroscopy.
